(19) United States
(12) Patent Application Publication
(10) Pub. No.: US 2025/0177366 A1
(43) Pub. Date: Jun. 5, 2025

(54) COMPOSITIONS AND METHODS FOR ALLEVIATING PAIN AND REDUCING ADVERSIVENESS OF STIMULI

(71) Applicant: UNIVERSITY OF WASHINGTON, Seattle, WA (US)

(72) Inventor: Ajay Dhaka, Seattle, WA (US)

(73) Assignee: UNIVERSITY OF WASHINGTON, Seattle, WA (US)

(21) Appl. No.: 18/683,338

(22) PCT Filed: Aug. 11, 2022
(86) PCT No.: PCT/US2022/074863

§ 371 Date: Feb. 13, 2024

Related U.S. Application Data
(60) Provisional application No. 63/233,121, filed on Aug. 13, 2021.

(51) Int. Cl. A61K 31/4409; A61P 29/00

(52) U.S. Cl. CPC A61K 31/4409; A61P 29/00

(57) ABSTRACT

Use of 4-(Propan-2-yl)-N-(pyridin-4-yl)benzamide (C15H16N2O) and similar compounds to treat pain and reduce the aversiveness of negative stimuli is described. The treatments can alleviate pain, anxiety, and depression. Such treatment may reduce the perception of pain or experience of an aversive reaction to a stimulus. The treatments can activate particular portions of the brain region, such as a subpallium or a telencephalic region.

### CROSS REFERENCE TO RELATED APPLICATION

This application is a U.S. National Phase Patent Application based on International Patent Application No. PCT/US2022/074863, filed on Aug. 11, 2022 which claims priority to U.S. Provisional Patent Application No. 63/233,121 filed Aug. 13, 2021, the entire contents each of which are incorporated by reference herein in their entirety.

### STATEMENT REGARDING FEDERALLY SPONSORED RESEARCH OR DEVELOPMENT

This invention was made with government support under Grant No. NS096635 awarded by the National Institutes of Health. The government has certain rights in the invention.

### FIELD OF THE DISCLOSURE

The current disclosure provides use of 4-(Propan-2-yl)-N-(pyridin-4-yl)benzamide (C15H16N2O) and similar compounds to treat pain and reduce the aversiveness of negative stimuli.

### BACKGROUND OF THE DISCLOSURE

Pain is of epidemic proportions in America, with 83 million adults living with pain that affects their participation in daily activities, and 75 million people with chronic debilitating pain. For example, as many as 1 in 3 adults in the United States currently suffer from chronic joint symptoms or arthritis. Osteoarthritis (OA) and rheumatoid arthritis (RA) are two of the common forms, in which 21 million and 2.1 million adults are affected by OA and RA, respectively, in the United States. Low back pain can cost the nation an estimated $27 billion annually in medical claims, and the same in disability payments and lost productivity. Despite the wide spread diseases or conditions, therapeutic options are limited and effectiveness of therapies remains insufficient.

Pain is both a sensory and affective experience (Price, Science 288, 1769-1772 (2000)). The unpleasant percept that dominates the affective dimension of pain is coupled with the motivational drive to engage protective behaviors that limit exposure to noxious stimuli (Baliki et al., Neuron 87, 474-491 (2015)).

Pain, and in particular chronic pain and depression are often co-morbid diseases. Depression is a condition that affects physical and mental health and is a leading cause of death and disability worldwide.

Anxiety disorders are among the most common mental health disorders, affecting 40 million American adults age 18 years and older (18%) in a given year (Kessler et al. Arch. Gen. Psych 2005). They generally last at least six months and can get worse if not treated. While the cause is not clear, they are believed to have both biological, social and psychological components ranging from heredity, personality, life experiences including reactions to stress such as traumatic events, and brain chemistry such as low neurotransmitter levels and problems with amygdala functioning. Anxiety disorders can result in persistent and disabling psychological and physiological symptoms that interfere with the day to day life of an affected individual and include disorders such as acute stress disorder, panic disorder, generalized anxiety disorder, agoraphobia with or without panic disorder, specific phobia, social phobia, obsessive-compulsive disorder, separation anxiety disorder, and post-traumatic stress disorder.

Post-Traumatic Stress Disorder (PTSD) and acute stress disorder (ASD) are anxiety disorders that can develop after exposure to a terrifying event or ordeal in which grave physical harm occurred or was threatened. Traumatic events that trigger PTSD or ASD include traumatic brain injury (TBI). TBI itself can lead to a variety of anxiety disorders. It is estimated that the lifetime prevalence of PTSD in the U.S. is 8% of the U.S. population. The rate among former combat soldiers runs much higher.

### SUMMARY OF THE DISCLOSURE

The current disclosure provides use of 4-(Propan-2-yl)-N-(pyridin-4-yl)benzamide (C15H16N2O) (referred to herein as AS1) and similar compounds to treat pain and reduce the aversiveness of negative stimuli. The treatments can alleviate pain, anxiety, and depression as explained in more detail in the following disclosure.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIGS. 1A-1E. Identifying AS1, a compound that reverses sensitized thermal aversion, from a small molecule screen. Photograph of a 5 days post-fertilization (dpf) zebrafish larva in a single arena of a 32-well temperature choice assay plate (FIG. 1A left panel) Representative traces/motion tracking of a zebrafish larva when both sides of the plate are set to 28.5° C. (FIG. 1A middle panel). No preference is demonstrated. Representative traces of a zebrafish larva given the choice between 28.5° C. and 31.5° C. after incubation in allyl isothiocyanate (AITC) (FIG. 1A right panel). Preference for the 28.5° C. side is shown. Results from screening Plate 43 in the sensitized temperature preference assay (FIG. 1B). Fish in all conditions except Pool 3 significantly favored the 28.5° C. side of the arena, whereas fish incubated in Pool 3 significantly chose the 31.5° C. portion of the arena. N=50 larvae for the control condition, N=49 for the AITC only condition, N=64, 52, 57, 63, 63, 61, 59, 63, 54, 58 larvae for Pools 2-11, respectively. The *** denotes the significant difference between Pool 3 and the AITC condition. Demultiplexing Pool 45-3 (FIG. 1C). Fish in all conditions significantly preferred the 28.5° C. side, whereas fish in Pool F preferred the 37.5° C. side. N=52, 63, 63, 63, 64, 58, 61, 64, 62, 57 larva for control, AITC, and groups A-H. *** denotes significant difference between F and the AITC only condition. The chemical structure of AS1 (4-propan-2-yl-N-pyridin-4-ylbenzamide) (FIG. 1D). Comparison of AS1 with two other potential novel analgesics, AS 2 and AS 3 (FIG. 1E). N=29, 58, 64, 49, 63 larvae for control, AITC, and AS1-3 conditions, respectively. * denotes significant difference from DMSO-treated control fish; #denotes significant difference from AITC-treated fish. *p<0.05, **p<0.01, ***p<0.001. One-way ANOVA with Dunnett's multiple comparison test was performed in the data shown in FIGS. 1B and 1C. One-way ANOVA with Tukey's multiple comparisons test was performed in the data shown in FIG. 1E. For all choice experiments (FIGS. 1B, 1C, 1E), a one-sample t test was performed with a hypothetical mean of 50% to determine if fish were significantly choosing one side of the arena.

FIGS. 2A-2C. Probing the effects of AS1 using a non-sensitized thermal aversion assay. AS1 dose-response curve in the non-sensitized thermal preference assay (28.5 vs 37.5° C.) (FIG. 2A). At low concentrations, AS1 does not influence preference for 28.5° C., whereas intermediate concentrations (1.25 μM) eliminate preference for either zone and high concentrations (>2.5 μM) induce preference for the 37.5° C. zone. N=58, 58, 60, 63, 63, 62 for 0, 0.25, 0.5, 1.25, 2.5, and 5 μM AS1 conditions, respectively. Velocity data for experiments shown in FIG. 2A (FIG. 2B). In control conditions (0 μM AS1), larval zebrafish locomote at much greater velocities in the 37.5° C. zone than in the 28.5° C. zone. Interestingly, as the concentration of AS1 increases, this velocity difference switches-velocity increases in the 28.5° C. portion but decreases in the 37.5° C. portion. At an intermediate concentration, there is no difference between velocities at 28.5 and 37.5° C. * denotes significant differences in swimming velocities between the 28.5 and 37.5° C. zones for the same group of fish, + denotes significant difference from the 0 μM AS1 37.5° C. swimming velocity, and #denotes significant difference from the 0 μM AS1 28.5° C. swimming velocity. Thermal preference assay (28.5° C. vs 37.5° C.) utilizing nine different structural analogs of AS1 (FIG. 2C). While two analogs slightly decreased the preference for 28.5° C., only one of them (9089110) abolished preference for either side. N=84, 59, 58, 46, 59, 55, 53, 32, 61, 59 fish for DMSO, 5538018, 7301738, 7755382, 7947669, 9047736, 9089110, 9089180, 9090934, and 9098532 conditions. A two-way ANOVA with Tukey's multiple comparisons test was performed on the data in FIG. 2B. A one-way ANOVA with Tukey's multiple comparisons test was performed on the data shown in FIG. 2C. For thermal preference assays, a one-sample t test was performed with a hypothetical mean of 50% to determine if fish were significantly choosing one side of the arena over the other.

FIGS. 3A-3F. AS1 can reverse the valence of other sensory modalities. Chemical attraction/aversion assay (FIG. 3A). When the experimental agarose is infused with 2% DMSO (dashed lines) both vehicle- and 5 μM AS1-treated fish demonstrate no preference for either end of the square chamber. When AITC (100 mM) is infused into the agarose (solid lines), the vehicle-treated fish are repelled by the agarose whereas the AS1-treated fish are attracted to the stimulus. N=25 fish per condition. The distance of each individual larva from the agarose at the final time point (t=800 s) of graph in FIG. 3A (FIG. 3B). In experiments with plain agarose, both AS1 and vehicle-treated fish are dispersed evenly throughout the arena. In experiments with AITC-infused agarose, vehicle-treated larval zebrafish are found significantly farther from the agarose while AS1-treated fish are clustered close to the AITC source. AITC aversion assay assessing multiple concentrations of AS1 (FIG. 3C). Fish exposed to low concentrations of AS1 (e.g. 1 μM) continue to avoid the AITC source, whereas fish exposed to a higher concentrations swim towards the AITC source. N=83 larvae for DMSO, 42 for 1 μM, 33 for 2.5 μM, and 37 for 5 μM AS1. Percentage of fish on the light side of a light/dark preference assay at 30 second intervals during a trial period of four minutes, averaged across all five trials (FIG. 3D). Vehicle-treated fish strongly prefer (i.e., quickly swim towards) the light half of the arena, whereas fish treated with 2.5 μM AS1 quickly navigate towards the dark. N=40 larvae per condition. Same experiment as the experiment conducted for results shown in FIG. 3D but showing the percentage of fish found on the light side of the arena for the last two minutes of each four-minute trial, averaged across the last four trials (FIG. 3E). While fish treated with vehicle and 1 μM AS1 significantly prefer the light side of the arena, fish treated with 2.5 μM significantly prefer the dark. Gradient phototaxis assay in which larval zebrafish were given the choice between a bright white background (shade 1) and increasingly darker shades (shades 2-7) (FIG. 3F). As the intensity of darkness increases, control zebrafish increasingly prefer shade 1, whereas 2.5 μM AS1-treated zebrafish increasingly prefer the contrasting dark shade. N=72 for DMSO and 81 for AS1 (1v2), 73 for DMSO and 78 for AS1 (1v3), 80 for DMSO and 70 for AS1 (1v4), 79 for DMSO and 79 for AS1 (1v5), 79 for DMSO and 77 for AS1 (1v6), 76 for DMSO and 77 for AS1 (1v7). *p<0.05, **p<0.01, ***p<0.001. One-way ANOVA with Tukey's multiple comparison test was performed in data shown in FIGS. 3B and 3E. Two-way ANOVA with Tukey's multiple comparisons test was performed in data shown in FIGS. 3C and 3F. For all temperature and light/dark choice experiments, a one-sample t test was performed with a hypothetical mean of 50% to determine if fish were significantly choosing one side of the arena.

FIGS. 4A-4D. AS1 does not appear to act upon canonical valence or pain relief circuitry. Temperature choice assay (28.5° C. vs 37.5° C.) with naloxone, a mu opioid receptor (MOR) antagonist (FIG. 4A). Naloxone did not appear to replicate or attenuate the effects of AS1; both control and naloxone only treated fish significantly chose the 28.5° C. side of the arena, whereas AS1 only and Naloxone+AS1-treated fish significantly chose the 37.5° C. side. N=37, 56, 52, and 45 fish for DMSO, 1 μM Naloxone, 5 μM AS1, and 1 μM Naloxone+5 μM AS1 conditions, respectively. Temperature choice assay (28.5° C. vs 37.5° C.) with various concentrations of caffeine, an anxiogenic stimulant (FIG. 4B). At all concentrations tested, larval zebrafish significantly chose the 28.5° C. side of the arena. N=52, 34, 51, 52, 34, and 38 fish for 0-515 mM caffeine, respectively. Temperature choice assay (28.5° C. vs 37.5° C.) with diazepam, a GABA-modulating anxiolytic drug (FIG. 4C). At all concentrations tested, larval zebrafish significantly chose the 28.5° C. side of the arena. N=115, 50, 53, 51, 49, 54, and 39 fish for 0-18 mM diazepam, respectively. Temperature choice assay (28.5° C. vs 37.5° C.) with buspirone, a 5HT_{1A} receptor agonist and anxiolytic drug (FIG. 4D). At all concentrations tested, larval zebrafish significantly preferred the 28.5° C. side of the arena. N=27, 43, 44, fish for 0, 10, and 50 μM buspirone conditions.*p<0.05, **p<0.01, ***p<0.001. Two-way ANOVA with Tukey's multiple comparisons test used in data shown in FIG. 4A. One-way ANOVA with Tukey's multiple comparisons test used in data shown in FIGS. 4B-4D. For all temperature choice experiments, a one-sample t test was performed with a hypothetical mean of 50% to determine if fish were significantly choosing one side of the arena.

FIGS. 5A-5C. The MOR antagonist naloxone does not replicate or reverse AS1-mediated attraction to noxious stimuli. AITC aversion assay (FIG. 5A). Naloxone alone did not affect AITC avoidance, and when co-applied with AS1 this drug did not reverse AS1-induced attraction towards AITC. N=80 fish for the DMSO condition, 71 for 5 μM AS1, 80 for 1 μM Naloxone, and 70 for 1 μM Naloxone+5 μM AS1. Phototaxis assay (FIG. 5B). All non-AS1-treated fish significantly chose the light side of the arena, whereas all AS1-treated fish significantly preferred the dark, regardless of whether 1 μM naloxone was applied. N=39 fish for the DMSO condition, 41 fish for the 2.5 μM AS1 condition, 41 fish for 1 μM naloxone, and 39 fish for 1 μM naloxone+2.5 μM AS1. The percentage of fish in the light at 30 second intervals in the four-minute trials of the phototaxis assay conducted for results shown in FIG. 5B, averaged across the five trials (FIG. 5C). As shown, AS1-treated fish quickly migrated to the dark half of the arena at the onset of each trial, whereas fish that did not receive AS1 migrated to the light half of the arena, regardless of whether naloxone had been applied. *p<0.05, **p<0.01, ***p<0.001. Two-way ANOVA with Tukey's multiple comparisons test used in b. To determine if fish were significantly choosing one side of the arena over the other a one-sample t test was performed with a hypothetical mean of 50%.

FIGS. 6A-6E. The 5HT_{2B}, sigma-1, and melanocortin-4 receptors do not appear to mediate the effects of AS1. Thermal preference assay (28.5° C. vs 37.5° C.) with the 5HT_{2B} agonist BW723C86 (FIG. 6A). All AS1-treated fish significantly preferred the 37.5° C. half of the arena, regardless of whether BW723C86 was co-applied. All conditions that did not receive AS1 significantly chose the 28.5° C. side. At no concentration did BW723C86 influence AS1-induced attraction towards noxious heat. N=46 fish for vehicle-treated controls, 43 for 1 μM BW723C86, 53 for 5 μM BW723C86, 53 for 10 μM BW723C86, 56 for 5 μM AS1, 50 for 1 μM BW723C86+5 μM AS1, 52 for 5 μM BW723C86+5 μM AS1, and 59 for 10 μM BW723C86+5 μM AS1. Thermal preference assay (28.5° C. vs 37.5° C.) with the 5HT_{2B} antagonist LY266097 (FIG. 6B). All AS1-treated fish significantly preferred the 37.5° C. half of the arena, regardless of whether LY266097 was co-applied. All conditions that did not receive AS1 significantly chose the 28.5° C. side. At no concentration did LY266097 influence AS1-induced attraction towards noxious heat. N=58 larvae for the DMSO condition, 57 for 1 μM LY266097, 50 for 5 μM LY266097, 50 for 10 μM LY266097, 59 for 5 μM AS1, 61 for 1 μM LY266097+5 μM AS1, 59 for the 5 μM LY266097+5 μM AS1, and 56 for the 10 μM LY266097+5 μM AS1. Thermal preference assay (28.5° C. vs 37.5° C.) with the Sigma-1 receptor agonist PRE-084 (FIG. 6C). All AS1-treated fish significantly preferred the 37.5° C. half of the arena, regardless of whether PRE-084 was co-applied. All conditions that did not receive AS1 significantly chose the 28.5° C. side. At no concentration did PRE-084 influence AS1-induced attraction towards noxious heat, despite potentiating preference for the 28.5° C. zone. N=90 fish for the DMSO condition, 27 for 1 μM PRE-084, 31 for 5 μM PRE-084, 25 for 10 μM PRE-084, 37 for 5 μM AS1, 40 for 1 μM PRE-084+5 μM AS1, 43 for 5 μM PRE-084+5 μM AS1, and 46 for 10 μM PRE-084+5 μM AS1. Thermal preference assay (28.5° C. vs 37.5° C.) with the Sigma-1 receptor antagonist BD1063 (FIG. 6D). All AS1-treated fish significantly preferred the 37.5° C. half of the arena, regardless of whether BD1063 was co-applied. All conditions that did not receive AS1 significantly chose the 28.5° C. side. At no concentration did BD1063 influence AS1-induced attraction towards noxious heat. N=30 for the DMSO condition, 26 for 1 μM BD1063, 30 for 5 μM BD1063, 28 for 10 μM BD1063, 27 for 5 μM AS1, 30 for 1 μM BD1063+5 μM AS1, 29 for 5 μM BD1063+5 μM AS1, and 28 for 10 μM BD1063+5 μM AS1. Thermal preference assay (28.5° C. vs 37.5° C.) with the melanocortin 4 receptor antagonist ML00253764 (FIG. 6E). All AS1-treated fish significantly preferred the 37.5° C. half of the arena, regardless of whether ML00253764 was co-applied. All conditions that did not receive AS1 significantly chose the 28.5° C. side. At no concentration did ML00253764 reduce AS1-induced attraction towards noxious heat, even though one concentration (5 μM) did slightly potentiate preference towards the 28.5° C. zone. N=30 fish for the DMSO condition, 31 for 1 μM ML00253764, 28 for 5 μM ML00253764, 31 for 10 μM ML00253764, 31 for 5 μM AS1, 32 for 1 μM ML00253764+5 μM AS1, 32 for 5 μM ML00253764+5 μM AS1, and 31 for 10 μM ML00253764+5 μM AS1. *p<0.05, **p<0.01, ***p<0.001. Two-way ANOVA with Sidak's multiple comparisons test used for data shown in FIG. 6A; two-way ANOVA with Tukey's multiple comparisons test used for data shown in FIGS. 6B-6E. To determine if fish were significantly choosing one side of the arena over the other a one-sample t test was performed with a hypothetical mean of 50%.

FIGS. 7A-7D. AS1 is not inherently attractive. A chemical attraction/aversion assay in which either vehicle (2% DMSO) or 50 mM AS1 was infused into agarose lining one side of a square petri dish (FIG. 7A). As shown, larval zebrafish were not drawn towards the AS1 source. N=20 larvae in the DMSO group, 15 in the AS1 group. Chemical attraction/aversion assay in which 2 mL of either 1% DMSO or 25 μM SKF was deposited into a trough on one side of a 3D-printed square arena (FIG. 7B). As shown, larval zebrafish exhibited a slight preference towards the side with SKF. N=40 larvae for the DMSO condition, and 43 for the 25 μM SKF condition. Chemical attraction/aversion assay in which 2 mL of 1% DMSO or variable concentrations of AS1 (in 1% DMSO vehicle) (FIG. 7C). Larval zebrafish did not appear to swim towards the AS1 source at any concentration tested for the duration of the assay. N=36 fish for the DMSO condition, 36 for the 10 μM AS1 condition, 36 for the 25 μM AS1 condition, 40 for the 50 μM AS1 condition, and 40 for the 100 μM AS1 condition. Experiment same as that conducted for results shown in FIG. 7C, but with 10% DMSO or 1 mM AS1 (in 10% DMSO) (FIG. 7D). N=36 larvae in both conditions.

FIGS. 8A, 8B. AS1 engages central nervous system circuitry in the presence of noxious stimuli. Number of photoconverted trigeminal neurons in control or AS1-treated zebrafish exposed to either 28.5° C. or 37.5° C. N=4 larvae per condition (FIG. 8A). Representative images of confocal z-stacks (maximum intensity projections) taken of whole brains of larval zebrafish stained for pERK (red) and tERK (green) (FIG. 8B). *p<0.05, **p<0.01, ***p<0.001. One-way ANOVA with Tukey's multiple comparisons test used in FIG. 8A.

FIGS. 9A-9F. The D1 receptor antagonist SCH23390 partially reverses AS1-induced attraction to noxious stimuli. Temperature choice assay (28.5° C. vs 37.5° C.) with SCH23390 (FIG. 9A). Control fish and those treated with 10 μM SCH significantly preferred the 28.5° C. side of the arena, whereas fish treated with 5 μM AS1 alone or 5 μM SCH+5 μM AS1 significantly preferred the 37.5° C. side. Interestingly, fish treated with 10 μM SCH+5 μM AS1 exhibited no preference between the two sides of the arena, indicating an abolition of choice following D1 receptor blockade. (Fish treated with 5 μM SCH alone also demonstrated no significant choice, but this may be due to a low N.) N=74 fish for the DMSO condition, 12 fish for 5 μM SCH, 43 fish for 10 μM SCH, 81 fish for 5 μM AS1, 52 fish for 5 μM SCH+5 μM AS1, and 50 for 10 μM SCH+5 μM AS1. Velocity data from the experiment conducted for the data shown in FIG. 9A (FIG. 9B). While AS1 treatment still significantly reduced swimming velocity at 37.5° C. and increased velocity at 28.5° C., treatment with progressively higher concentrations of SCH significantly reduced this AS1-induced heightened velocity at 28.5° C., and at 10 μM SCH (+5 μM AS1), there is no longer a significant difference in swimming velocity between the 28.5° C. and 37.5° C. zones. * denotes significant differences in swimming velocities between the 28.5 and 37.5° C. zones for the same group of fish, + denotes significant difference from the 0 μM AS1 37.5° C. swimming velocity, and #denotes significant difference from the 0 μM AS1 28.5° C. swimming velocity. AITC aversion assay (FIGS. 9C and 9D). SCH alone did not elicit changes in AITC avoidance, whereas concentrations of SCH 5 μM and above partially attenuate AS1-induced attraction to AITC. N=44 fish for DMSO, 38 for 1 μM SCH, 34 for 5 μM SCH, 39 for 10 μM SCH, 37 for 20 μM SCH, 14 for 5 μM AS1, 43 for 1 μM SCH+5 μM AS1, 33 for 5 μM SCH+5 μM AS1, 41 for 10 μM SCH+5 μM AS1, 41 for 20 μM SCH+5 μM AS1. Distance of each individual larva from the AITC stimulus at the final time point (840 s) for the experiment conducted for the results shown in FIGS. 9C and 9D (FIG. 9E). Phototaxis assay (FIG. 9F). While SCH alone caused a significant decrease in the percentage of fish found in the light half of the arena when applied at higher concentrations, fish in these conditions still significantly preferred the light half of the arena. When co-applied with AS1, 15 μM and 20 μM SCH were able to significantly reduce AS1-induced preference for the dark, with 15 μM SCH abolishing preference entirely. N=80 fish for DMSO, 80 for 10 μM SCH, 40 for 15 μM SCH, 80 for 20 μM SCH, 80 for 2.5 μM AS1, 40 for 10 μM SCH+2.5 μM AS1, 40 for 15 μM SCH+2.5 μM AS1, and 40 for 20 μM SCH+2.5 μM AS1. *p<0.05, **p<0.01, *** p<0.001. Two-way ANOVA with Tukey's multiple comparisons test used in data shown in FIGS. 9A, 9B, 9F. For all temperature and light/dark choice experiments, a one-sample t test was performed with a hypothetical mean of 50% to determine if fish were significantly choosing one side of the arena over the other.

FIGS. 10A-10F. The D1 receptor agonist SKF82958 partially reverses AS1-induced attraction to noxious stimuli. Temperature choice assay (28.5° C. vs 37.5° C.) with various concentrations of SKF82958 (FIG. 10A). DMSO or SKF alone treated fish demonstrated significant preference for the 28.5° C. side of the arena, with the highest concentration of SKF tested eliciting a slight potentiation of preference for the 28.5° C. side. While all AS1-treated fish still preferred the 37.5° C. side of the arena regardless of whether SKF was also present, application of both 10 μM and 30 μM SKF induced a significant decrease in the proportion of time fish spent in the 37.5° C. half of the arena. N=53 fish for the DMSO condition, 53 for 10 μM SKF, 54 for 30 μM SKF, 61 for 5 μM AS1, 60 for 10 μM SKF+5 μM AS1, and 60 for 30 μM SKF+5 μM AS1. Swimming velocity from the experiment conducted for the results shown in FIG. 10A (FIG. 10B). While AS1 treatment still reversed velocity patterns observed in the 37.5° C. and 28.5° C. zones, co-incubation with SKF partially attenuated this effect. At 10 μM SKF, the difference between swimming velocity in both zones was insignificant, and the swimming velocity at 28.5° C. was no different than that of vehicle-treated control fish. * denotes significant differences in swimming velocities between the 28.5 and 37.5° C. zones for the same group of fish, + denotes significant difference from the 0 μM AS1 37.5° C. swimming velocity, and #denotes significant difference from the 0 μM AS1 28.5° C. swimming velocity. AITC aversion assay (FIGS. 10C and 10D). SKF alone did not elicit any large changes in avoidance of the noxious AITC stimulus, but partially reversed AS1-induced attraction towards AITC. N=40 fish for the DMSO condition, 40 for the 10 μM SKF, 40 for the 30 μM SKF, 38 for 2.5 μM AS1, 36 for 10 μM SKF+2.5 μM AS1, and 39 for 30 μM SKF+2.5 μM AS1. Distance of each individual larva from the AITC source at the final time point (840 s) (FIG. 10E). While AS1-treated fish clustered near the AITC source, fish co-incubated in SKF exhibited greater dispersal across the arena by the end of the experiment. Phototaxis assay (FIG. 10F). While 10 μM SKF did elicit a slight decrease in the proportion of fish found in the light by itself, these fish still significantly preferred the light side, as did the vehicle-treated fish. AS1-only treated fish significantly preferred the dark half of the arena, but concurrent treatment with 10 μM SKF restored preference for the light side. N=40 for all conditions. *p<0.05, **p<0.01, ***p<0.001. Two-way ANOVA with Tukey's multiple comparisons test used in data shown in FIGS. 10A, 10B, and 10F. For all temperature and light/dark choice experiments, a one-sample t test was performed with a hypothetical mean of 50% to determine if fish significantly chose one side of the arena over the other.

FIGS. 11A-11F. The D2 receptor antagonist sulpiride did not replicate or reverse the effects of AS1 in multiple choice assays. Temperature choice assay (28.5° C. vs 37.5° C.) with various concentrations of sulpiride (FIG. 11A). All AS1-treated fish significantly preferred the 37.5° C. side, whereas fish treated only with sulpiride or vehicle solution significantly chose the 28.5° C. side. N=28 larvae for 1% DMSO, 37 for 1 μM sulpiride, 41 for 5 μM sulpiride, 44 for 10 μM sulpiride, 30 for 30 μM sulpiride, 58 for 5 μM AS1, 49 for 1 μM sulpiride+5 μM AS1, 52 for 5 μM sulpiride+5 μM AS1, 54 for 10 μM sulpiride+5 μM AS1, and 62 for 30 μM sulpiride+5 μM AS1. Swimming velocity of the fish in the experiment conducted for the results shown in FIG. 11A (FIG. 11B). AS1-treated fish had significantly lower velocities at 37.5° C. than at 28.5° C. at all concentrations of sulpiride tested apart from 1 μM. Additionally, AS1-treated fish had significantly lower swimming velocities at 37.5° C. than non-AS1 treated fish at all concentrations of sulpiride tested. * denotes significant differences in swimming velocities between the 28.5 and 37.5° C. zones for the same group of fish, + denotes significant difference from the 0 μM AS1 37.5° C. swimming velocity, and #denotes significant difference from the 0 μM AS1 28.5° C. swimming velocity. AITC aversion assay with various concentrations of sulpiride (FIG. 11C). None of the sulpiride concentrations tested elicited a change in AITC aversion. N=174 fish for DMSO, 36 fish for 1 μM sulpiride, 34 fish for 5 μM sulpiride, 177 fish for 10 μM sulpiride, and 33 fish for 30 μM sulpiride. AITC aversion assay for various concentrations of sulpiride+5 μM AS1, with the same DMSO control fish (0 μM Sulp) used in the experiment conducted for the results shown in FIG. 11C (FIG. 11D). At no concentration tested does sulpiride attenuate AS1-induced attraction to AITC. N=169 fish for 5 μM AS1, 32 fish for 1 μM sulpiride+5 μM AS1, 32 fish for 5 μM sulpiride+5 μM AS1, 172 fish for 10 μM sulpiride+5 μM AS1, and 33 fish for 30 μM sulpiride+5 μM AS1. The distance of each individual larva from the AITC-infused agarose at the final time point (t=840 s) of the experiment conducted for results shown in FIGS. 11C and 11D (FIG. 11E). Whereas control and all sulpiride-only condition fish localized farther from the AITC source, all AS1-treated fish congregated closer to the AITC source. Light/dark preference assay (FIG. 11F). No tested concentration of sulpiride was capable of reversing AS1-induced preference for the dark. N=39 fish for 0 μM sulpiride, 40 for 10 μM sulpiride, 40 for 30 μM sulpiride, 77 for 0 μM sulpiride+5 μM AS1, 79 for 10 μM sulpiride+5 μM AS1, and 83 for 30 μM sulpiride+5 μM AS1.* p<0.05, **p<0.01, ***p<0.001. Two-way ANOVA with Tukey's multiple comparisons test used in results shown in FIGS. 11A, 11B, and 11F. For all temperature and light/dark choice experiments, a one-sample t test was performed with a hypothetical mean of 50% to determine if fish were significantly choosing one side of the arena.

FIGS. 12A-12F. The D2 receptor agonist sumanirole maleate does not replicate or reverse the effects of AS1 in multiple assays. Temperature choice assay (28.5° C. vs 37.5° C.) with various concentrations of sumanirole (FIG. 12A). All AS1-treated fish significantly preferred the 37.5° C. side regardless of sumanirole concentration, whereas fish treated only with sumanirole or vehicle solution significantly chose the 28.5° C. side. N=44 fish for DMSO, 41 for 1 μM sumanirole, 59 for 5 μM sumanirole, 44 for 10 μM sumanirole, 48 for 30 μM sumanirole, 28 for 5 μM AS1, 46 for 1 μM sumanirole+5 μM AS1, 50 for 5 μM sumanirole+5 μM AS1, 37 for 10 μM sumanirole+5 μM AS1, and 53 for 30 μM sumanirole+5 μM AS1. Velocity data for fish in the experiment conducted for results shown in FIG. 12A (FIG. 12B). AS1 treatment significantly increased swimming velocity in the 28.5° C. zone and decreased swimming velocity in the 37.5° C. zone, regardless of the concentration of sumanirole co-applied to the zebrafish. Sumanirole alone had no effect upon swimming velocity in either zone. * denotes significant differences in swimming velocities between the 28.5 and 37.5° C. zones for the same group of fish, + denotes significant difference from the 0 μM AS1 37.5° C. swimming velocity, and #denotes significant difference from the 0 μM AS1 28.5° C. swimming velocity. AITC aversion assay (FIGS. 12C and 12D). Sumanirole alone did not affect aversion to this noxious chemical stimulus, but at higher concentrations partially attenuated AS1-induced attraction to AITC. N=68 for 0 μM sumanirole, 36 for 1 μM sumanirole, 36 for 5 μM sumanirole, 61 for 10 μM sumanirole, 63 for 30 μM sumanirole, 64 for 0 μM sumanirole+2.5 μM AS1, 34 for 1 μM sumanirole+2.5 μM AS1, 34 for 5 μM sumanirole+2.5 μM AS1, 61 for 10 μM sumanirole+2.5 μM AS1, and 63 for 30 μM sumanirole+2.5 μM AS1. Distance of each individual larva from the AITC stimulus at the final time point (840 s) from the experiment conducted for the results shown in FIGS. 12C and 12D (FIG. 12E). Phototaxis assay (FIG. 12F). AS1-treated fish significantly chose the dark half of the arena regardless of what concentration of sumanirole was co-applied, whereas vehicle and sumanirole-only treated fish significantly chose the light half of the arena. N=23 fish for 0 μM sumanirole, 37 for 1 μM sumanirole, 41 for 5 μM sumanirole, 40 for 10 μM sumanirole, 40 for 0 μM sumanirole+2.5 μM AS1, 46 for 1 μM sumanirole+2.5 μM AS1, 43 for 5 μM sumanirole+2.5 μM AS1, and 40 for 10 μM sumanirole+2.5 μM AS1. *p<0.05, **p<0.01, ***p<0.001. Two-way ANOVA with Sidak's multiple comparisons test used in data shown in FIGS. 12A and 12B. Two-way ANOVA with Tukey's multiple comparisons test used in data shown in FIG. 12F. For all temperature and light/dark choice experiments, a one-sample t test was performed with a hypothetical mean of 50% to determine if fish were significantly choosing one side of the arena over the other.

### DETAILED DESCRIPTION

Pain is of epidemic proportions in America, with 83 million adults living with pain that affects their participation in daily activities, and 75 million people with chronic debilitating pain. For example, as many as 1 in 3 adults in the United States currently suffer from chronic joint symptoms or arthritis. Osteoarthritis (OA) and rheumatoid arthritis (RA) are two of the common forms, in which 21 million and 2.1 million adults are affected by OA and RA, respectively, in the United States. Low back pain can cost the nation an estimated $27 billion annually in medical claims, and the same in disability payments and lost productivity. Despite the wide spread diseases or conditions, therapeutic options are limited and effectiveness of therapies remains insufficient.

Pain is both a sensory and affective experience (Price, Science 288, 1769-1772 (2000)). The unpleasant percept that dominates the affective dimension of pain is coupled with the motivational drive to engage protective behaviors that limit exposure to noxious stimuli (Baliki et al., Neuron 87, 474-491 (2015)).

Pain, and in particular chronic pain and depression are often co-morbid diseases. Depression is a condition that affects physical and mental health and is a leading cause of death and disability worldwide.

Anxiety disorders are among the most common mental health disorders, affecting 40 million American adults age 18 years and older (18%) in a given year (Kessler et al. Arch. Gen. Psych 2005). They generally last at least six months and can get worse if not treated. While the cause is not clear, they are believed to have both biological, social and psychological components ranging from heredity, personality, life experiences including reactions to stress such as traumatic events, and brain chemistry such as low neurotransmitter levels and problems with amygdala functioning. Anxiety disorders can result in persistent and disabling psychological and physiological symptoms that interfere with the day to day life of an affected individual and include disorders such as acute stress disorder, panic disorder, generalized anxiety disorder, agoraphobia with or without panic disorder, specific phobia, social phobia, obsessive-compulsive disorder, separation anxiety disorder, and post-traumatic stress disorder.

Post-Traumatic Stress Disorder (PTSD) and acute stress disorder (ASD) are anxiety disorder that can develop after exposure to a terrifying event or ordeal in which grave physical harm occurred or was threatened. Traumatic events that trigger PTSD or ASD include traumatic brain injury (TBI). TBI itself can lead to a variety of anxiety disorders. It is estimated that the lifetime prevalence of PTSD in the U.S. is 8% of the U.S. population. The rate among former combat soldiers runs much higher.

The current disclosure provides use of 4-(Propan-2-yl)-N-(pyridin-4-yl)benzamide (C15H16N2O) (referred to herein as AS1) and similar compounds to treat pain and reduce the aversiveness of negative stimuli. The treatments can alleviate pain, anxiety, and depression as explained in more detail below.

Aspects of the current disclosure are described as follows: (i) Compositions for Administration; (ii) Methods of Use; (iii) Exemplary Embodiments; (iv) Experimental Examples; and (v) Closing Paragraphs. These headings are provided for organizational purposes only and do not limit the scope or interpretation of the disclosure.

### (i) Compositions for Administration

AS1 and other compounds disclosed herein (collectively, active ingredients) can be formulated into compositions for administration to subjects. Salts and/or pro-drugs of active ingredients can also be used.

A pharmaceutically acceptable salt includes any salt that retains the activity of an active ingredient and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes active ingredients or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration. For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the composition can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot compositions can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of nanoparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition: at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, compositions and formulations can be prepared to meet sterility, pyrogenicity, general safety, and purity standards as required by U.S. FDA Office of Biological Standards and/or other relevant foreign regulatory agencies.

### (ii) Methods of Use

Methods disclosed herein include treating subjects. Subjects include, e.g., humans, veterinary animals (dogs, cats, reptiles, birds) livestock (e.g., horses, cattle, goats, pigs, chickens) and research animals (e.g., monkeys, rats, mice, fish). Treating subjects includes delivering therapeutically effective amounts. Therapeutically effective amounts include those that provide effective amounts, prophylactic treatments and/or therapeutic treatments.

An “effective amount” is the amount of a composition or formulation necessary to result in a desired physiological change in the subject. Effective amounts are often administered for research purposes. Effective amounts disclosed herein can cause a statistically-significant effect in an animal model or in vitro assay relevant to the assessment of a condition's development, progression, and/or resolution.

A “prophylactic treatment” includes a treatment administered to a subject who does not display signs or symptoms of a condition or displays only early signs or symptoms of a condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment, or therapeutic treatment are not mutually exclusive, and in particular embodiments, administered dosages may accomplish more than one treatment type.

In certain examples, the condition to be researched, diagnosed, or treated includes pain or a pain-related disorder. Pain-related disorders may be a chronic or an acute disease. Pain-related disorders include those due to an illness, injury, surgery, trauma, medical procedure, infection, exposure to a toxin, or the like. Pain-related disorders include cancer, fibromyalgia, arthritis, complex regional pain syndrome, cluster headaches, sciatica, Parkinson's disease, Lyme disease, Shingles, appendicitis, kidney stones, pancreatitis, gout, endometriosis, trigeminal neuralgia, Sickle cell disease, HIV-related neuropathic pain, multiple sclerosis, spinal cord injury, diabetic neuropathy, herpetic neuralgia, and more. Pain-related disorders may be rated on a pain scale which may be numerical, visual, or categorical scales. Pain scales may be quantitative or qualitative. Examples of pain scales include the McGill Pain Scale, Mankoski Pain Scale, and Descriptor Differential Scale of Pain Intensity, Wong-Baker Faces Scale, FLACC (Face, Legs, Arms, Crying, Consolability) Scale, COMFORT Scale, Critical Care Pain Observation Tool (CPOT), Defense and Veterans Pain Rating Scale (DVPRS), Behavioral Pain Scale (BPS), and Pain Assessment in People with Dementia (PAINAD).

Types of pain that can be treated include musculoskeletal pain, nerve related pain, arthritis, ulnar deviation, boutonniere deformity, swan neck deformity, z-thumb deformity, subluxation at the metacarpophalangeal joint; inflammatory arthropathies, such as, ankylosing spondylitis, psoriatic arthritis, Reiter's syndrome, acute gout, dystocia, postpartum lochiostasis, abdominal pain, and metastatic bone pain. Other pain related conditions include cancer related pain, vascular pain, Raynaud's disease, psychogenic pain, trigeminal neuralgia, spinal cord injury, spasticity, post dural puncture headache, pelvic pain, head and neck cancer pain, complex regional pain syndrome, postherpetic neuralgia (shingles), peripheral neuralgia, nerve injuries, phantom limb pain, pelvic and urogenital pain, post-traumatic pain, post-amputation pain, temporomandibular disorders, and AIDS-related pain.

Examples of pains due to sports injury, strain or inflammation of tendons or ligaments include tennis elbow, frozen shoulder, carpal tunnel syndrome, plantar fasciitis, and Achilles tendonitis.

In certain examples, compositions are administered to a subject before the subject is expected to pain. A subject could be expected to experience pain before a medical procedure or treatment (e.g., surgery or chemotherapy), before childbirth, before an endurance athletic event, before a physical training exercise of a higher intensity than the subject is accustomed, before exposure to a stressful stimulus, or the like.

In certain examples, treatments disclosed herein result in “hypoalgesia”, a decreased sensitivity to pain.

A variety of screening methods may be used for assessing whether a composition relieves pain and/or reduces pain affective-motivational behavior including sensory perception of pain, pain avoidance behavior, hyperalgesia, and allodynia.

“Hyperalgesia” refers to an abnormally increased sensitivity to pain, including pain that results from excessive sensitivity to stimuli. Hyperalgesia can result from damage to nociceptors or nerves. Primary hyperalgesia refers to pain sensitivity that occurs in damaged tissues.

Secondary hyperalgesia refers to pain sensitivity that occurs in undamaged tissue surrounding damaged tissue. Examples of hyperalgesia include, without limitation, thermal hyperalgesia (i.e., hypersensitivity to cold or heat) and opioid-induced hyperalgesia (e.g., hypersensitivity to pain as a result of long-term opioid use such as caused by treatment of chronic pain).

“Allodynia” means pain that results from a normally non-painful, non-noxious stimulus to the skin or body surface. Examples of allodynia include, but are not limited to, thermal (hot or cold) allodynia (e.g., pain from normally mild temperatures), tactile or mechanical allodynia (e.g., static mechanical allodynia (pain triggered by pressure), punctate mechanical allodynia (pain when touched), or dynamic mechanical allodynia (pain in response to stroking or brushing)), movement allodynia (pain triggered by normal movement of joints or muscles), and the like.

Testing for hyperalgesia or dynamic mechanical allodynia and may include, for example, brushing the skin of a subject with a cotton ball or paintbrush. Punctate mechanical allodynia and hyperalgesia can be tested, for example, with a pinprick or von Frey filaments of varying forces (0.08-2940 mN). Static hyperalgesia can be tested, for example, by applying pressure to the skin or underlying tissue by pressing a finger or using a pressure algometer.

Additional methods include stimulus-evoked behavioral tests such as a mechanical withdrawal test, an electronic Von Frey test, a manual Von Frey test, a Randall-Selitto test, a Hargreaves test, a hot plate test, a cold plate test, a thermal probe test, an acetone evaporation test, cold plantar test, and a temperature preference test; and non-stimulus-evoked behavioral tests such as a grimace scale test, weight bearing and gait analysis, locomotive activity test (e.g., still, walking, trotting, running, distance traveled, velocity, and eating/drinking).

Certain examples, treat “pathological pain”, which refers to pain resulting from a pathology, such as from functional disturbances and/or pathological changes, lesions, burns and the like. One form of pathological pain is “neuropathic pain” which is pain thought to initially result from nerve damage but extended or exacerbated by other mechanisms including glial cell activation. Examples of pathological pain include thermal or mechanical hyperalgesia, thermal or mechanical allodynia, diabetic pain, pain arising from irritable bowel or other internal organ disorders, endometriosis pain, phantom limb pain, complex regional pain syndromes, fibromyalgia, low back pain, cancer pain, pain arising from infection, inflammation or trauma to peripheral nerves or the central nervous system, multiple sclerosis pain, entrapment pain, and the like.

In particular embodiments, administration of compositions provides “analgesia”, defined herein as the relief of pain without the loss of consciousness.

In certain examples, the condition to be researched, diagnosed, or treated includes an aversive reaction to a stimulus. In certain examples, the aversive reaction to a stimulus creates a condition, such as an anxiety-related disorder, depression, acute stress disorder, and/or post-traumatic stress disorder (PTSD).

The expression “treating anxiety” in this context refers to an improvement in anxiety symptoms, where the improvement may be characterized qualitatively or quantitatively by assessments known in the art. Examples of types of anxiety include generalized anxiety disorder, social anxiety disorder, panic disorder, specific phobias, and post-traumatic stress disorder. Anxiety may be measured by an anxiety rating scale, for example, State-Trait Anxiety Inventory (STAI), the Fear Survey Schedule, Beck Anxiety Inventory (BAI), Brief Fear of Negative Evaluation Scale—BFNE, Clinician Administered PTSD Scale (CAPS), Daily Assessment of Symptoms—Anxiety, Generalized Anxiety Disorder 7 (GAD-7), Hamilton Anxiety Scale (HAM-A), Hospital Anxiety and Depression Scale (HADS-A), Leibowitz Social Anxiety Scale (LSAS), Overall Anxiety Severity and Impairment Scale (OASIS), Panic and Agoraphobia Scale (PAS), Panic Disorder Severity Scale (PDSS), PTSD Symptom Scale—Self-Report Version, Social Phobia Inventory (SPIN), Trauma Screening Questionnaire, Yale-Brown Obsessive Compulsive Scale (Y-BOCS), and the Zung Self-Rating Anxiety Scale.

Symptoms of anxiety disorders may vary depending on the disorder, but may include feelings of panic; persistent worry; doubt; dread; fear; uneasiness; uncontrollable, obsessive thoughts; repeated thoughts or flashbacks of traumatic experiences; mood instability; agitation; restlessness; dyspepsia; headaches; dyspnea; nightmares; ritualistic behaviors, such as repeated hand washing; insomnia; cold or sweaty hands and/or feet; shortness of breath; palpitations; an inability to be still and calm; intense startle reflex; dry mouth; numbness or tingling in the hands or feet; nausea; muscle tension; and/or dizziness.

Panic Disorder is characterized by sudden attacks of intense fear or anxiety, usually associated with numerous physical symptoms such as heart palpitations, rapid breathing or shortness of breath, blurred vision, dizziness, and racing thoughts. Generalized anxiety disorder is evidenced by general feelings of anxiety such as mild heart palpitations, dizziness, and excessive worry. Agoraphobia is the anxiety of being in places where escape might be difficult or embarrassing or in which help may not be available should a panic attack develop. Phobias result in extreme anxiety and/or fear associated with the object or situation of avoidance. Obsessive compulsive disorders are characterized by persistent, often irrational, and seemingly uncontrollable thoughts and actions which are used to neutralize the obsessions.

“Acute stress disorder (ASD)” is an anxiety disorder that involves a reaction following exposure to a traumatic event or stressor (e.g., a serious injury to oneself, witnessing an act of violence, hearing about something horrible that has happened to someone one is close to). While similar to PTSD, the duration of symptoms of ASD is shorter than that for PTSD. For a diagnosis of ASD, the full range of symptoms may be present for two days to four weeks.

“Post-Traumatic Stress Disorder (PTSD)” is an anxiety disorder that can develop after exposure to a terrifying event or ordeal in which grave physical harm occurred or was threatened to oneself or others. Traumatic events that may trigger PTSD include violent personal assaults, natural or human-caused disasters, accidents, or military combat, all of which can involve traumatic brain injury (TBI). PTSD was described in veterans of the American Civil War, and was called “shell shock,” “combat neurosis,” and “operational fatigue.” PTSD symptoms can be grouped into three categories: (1) re-experiencing symptoms; (2) avoidance symptoms; and (3) hyperarousal symptoms. Exemplary re-experience symptoms include flashbacks (e.g., reliving the trauma over and over, including physical symptoms like a racing heart or sweating), bad dreams, and frightening thoughts. Re-experiencing symptoms may cause problems in a person's everyday routine. They can start from the person's own thoughts and feelings. Words, objects, or situations that are reminders of the event can also trigger re-experiencing. Symptoms of avoidance include staying away from places, events, or objects that are reminders of the experience; feeling emotionally numb; feeling strong guilt, depression, or worry; losing interest in activities that were enjoyable in the past; and having trouble remembering the dangerous event. Things that remind a person of the traumatic event can trigger avoidance symptoms. These symptoms may cause a person to change his or her personal routine. For example, after a bad car accident, a person who usually drives may avoid driving or riding in a car. Hyperarousal symptoms include being easily startled, feeling tense or “on edge”, having difficulty sleeping, and/or having angry outbursts. Hyperarousal symptoms are usually constant, instead of being triggered by things that remind one of the traumatic event. They can make the person feel stressed and angry. These symptoms may make it hard to do daily tasks, such as sleeping, eating, or concentrating. Therefore, generally, PTSD symptoms can include nightmares, flashbacks, emotional detachment or numbing of feelings (emotional self-mortification or dissociation), insomnia, avoidance of reminders and extreme distress when exposed to the reminders (“triggers”), loss of appetite, irritability, hypervigilance, memory loss (may appear as difficulty paying attention), excessive startle response, clinical depression, stress, and anxiety. The symptoms may last for a month, for three months, or for longer periods of time.

The expression “treating depression” refers to an improvement in symptoms associated with depression, where the improvement may be characterized qualitatively or quantitatively by assessments known in the art. The depression may be treatment resistant depression where the patient has previously been unresponsive to anti-depressant medication. Examples of types of depression or major depressive disorder include: depression with melancholic features or somatic syndrome, depression with psychotic features, depression with atypical features, depression with catatonic features, depression with anxious distress and depression with mixed features. Episodes of depression of any type may have an illness pattern such as single, recurrent, seasonal or persistent, and/or related to organic causation (such as medication-induced or caused by behavioral and psychological symptoms of dementia (BPSD)) or neuroendocrine disruption such as in pre-menstrual dysphoric disorder, peri-menopausal or perinatal (including antenatal and post-natal) depression. Depression may be measured by a depression rating scale, for example, Hamilton Rating Scale for Depression (HAM-D), Beck Depression Inventory (BDI), Beck Hopelessness Scale, Centre for Epidemiological Studies—Depression Scale (CES-D), Patient Health Questionnaire, Center for Epidemiological Studies Depression Scale for Children (CES-DC), Clinically Useful Depression Outcome Scale, Diagnostic Inventory for Depression, Edinburgh Postnatal Depression Scale (EPDS), Inventory of Depressive Symptomatology, Geriatric Depression Scale (GDS), Hospital Anxiety and Depression Scale, Kutcher Adolescent Depression Scale (KADS), Major Depression Inventory (MDI), Montgomery-Asberg Depression Rating Scale (MADRS), Mood and Feelings Questionnaire (MFQ), Zung Self-Rating Depression Scale, or Cornell Scale for Depression in Dementia (CSDD).

The treatment may reduce the symptoms of depression, anxiety and/or PTSD. For example, the treatment may allow that patient to perform daily tasks, such as showering, cleaning, shopping and planning for future events that had not been possible before treatment. Improvements in mood, libido, concentration may also occur on treatment.

In certain examples, compositions are administered to a subject when the subject is experiencing an aversive reaction to a stimulus. An aversive reaction to a stimulus can include a feeling of anxiety, nausea, discomfort, or fear. Aversive reaction to a stimulus can be self-reported, and can also be identified through the detection of physiological parameters, such as increased galvanic skin response, sweating, shaking, crying, cringing, hiding, etc.

In certain examples, compositions are administered to a subject before the subject is expected to experience an aversive reaction to a stimulus. A subject can be expected to experience an aversive reaction to a stimulus when the subject has experienced the stimulus before and previously experienced an aversive reaction to the stimulus. For example, if the subject has flight anxiety, the subject could be expected to have an aversive reaction to an upcoming flight. If the subject has a phobia, the subject could be expected to have an aversive reaction to an upcoming exposure to the cause of phobia. Common phobia are to heights, elevators, reptiles, spiders, flying, dentists, the sight of blood, crowds, darkness, pain, needles, injections, etc.

In certain examples, compositions are administered to a subject to positively impact the mood of the subject; for its ability to prevent, reduce or stop anxiety in the subject; for its ability to prevent, relieve or stop stress in the subject; for its ability to cause pleasurable effect on the subject; for its ability to induce or enhance pleasant feeling of the subject; and/or for its ability to prevent, relieve or stop one or more undesired sensations felt by the subject; ability to make the subject relax and/or feel comfortable; for its ability to prevent, relieve, or stop discomfort in the subject.

Certain methods of the disclosure include imaging. Imaging can include high-resolution fluorescence imaging, NIR fluorescence imaging, photoacoustic imaging, and/or image-guided surgery imaging. “Photoacoustic imaging” as used herein is a process of delivering light energy to cells or a tissue to cause a thermoelastic expansion in the cells or tissue that generates ultrasound waves that are then detected by a transducer to produce images of optical absorption contrast within the cells or tissues.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (iii) Exemplary Embodiments

1. A composition including:
   at least one of:
   4-(Propan-2-yl)-N-(pyridin-4-yl)benzamide,
   4-propyl-N-4-pyridinylbenzamide,
   4-isopropyl-N-3-pyridinylbenzamide, or
   N-(4-isopropylphenyl)-3,5-dimethyl-4-isoxazolecarboxamide; and
   a pharmaceutically acceptable carrier.
2. The composition of embodiment 1, wherein the composition includes the 4-(Propan-2-yl)-N-(pyridin-4-yl)benzamide.
3. The composition of embodiment 2, formulated for oral delivery or injection.
4. The composition of embodiment 3, wherein the composition formulated for oral delivery is in the form of a tablet, pill, dragee, capsule, liquid, gel, syrup, slurry, or suspension.
5. A method of treating a subject in need thereof including administering a therapeutically effective amount of the composition of any of embodiments 1-3 to the subject, thereby treating the subject in need thereof.
6. The method of embodiment 5, wherein the administering occurs when the subject is experiencing pain and the treating reduces the perception of pain.
7. The method of embodiment 5, wherein the administering occurs before the subject is expected to experience pain, and the treating reduces the expected experience of pain.
8. The method of any of embodiments 5-7, wherein the administering occurs when the subject is experiencing an aversive reaction to a stimulus and the treating reduces the aversive reaction to the stimulus.
9. The method of any of embodiments 5-8, wherein the administering occurs before the subject is expected to experience an aversive reaction to a stimulus, and the treating reduces the experience of the aversive reaction to the stimulus.
10. The method of any of embodiments 5-9, wherein the subject has anxiety or an anxiety-related disorder.
11. The method of any of embodiments 5-10, wherein the subject has depression.
12. The method of any of embodiments 5-11, wherein the subject has post-traumatic stress disorder.
13. The method of any of embodiments 5-12, wherein the subject is a mammal.
14. The method of any of embodiments 5-13, wherein the administering is oral, sublingual, subcutaneous, intravenous, intradermal, intraarterial, intranodal, intralymphatic, intraperitoneal, intralesional, topical, or intramuscular.
15. A method of activating a portion of a brain region in a subject including administering a therapeutically effective amount of the composition of any of embodiments 1-3 to the subject, thereby activating the portion of the brain region.
16. The method of embodiment 15, wherein the administering occurs when the subject is experiencing pain.
17. The method of embodiment 15, wherein the administering occurs before the subject is expected to experience pain.
18. The method of any of embodiments 15-17, wherein the subject is experiencing an aversive reaction to a stimulus.
19. The method of any of embodiments 15-18, wherein the administering occurs before the subject is expected to experience an aversive reaction to a stimulus.
20. The method of any of embodiments 15-19, wherein the subject has anxiety or an anxiety-related disorder.
21. The method of any of embodiments 15-20, wherein the subject has depression.
22. The method of any of embodiments 15-21, wherein the subject has post-traumatic stress disorder.
23. The method of any of embodiments 15-22, wherein the subject is a mammal.
24. The method of any of embodiments 15-23, wherein the administering is oral, sublingual, subcutaneous, intravenous, intradermal, intraarterial, intranodal, intralymphatic, intraperitoneal, intralesional, topical, or intramuscular.
25. The method of any of embodiments 15-24, wherein the brain region is a subpallium or within a telencephalic region.
26. The method of embodiment 25, wherein the brain region within the telencephalic region is a basal ganglia.
27. The method of embodiments 25 or 26, wherein the brain region within the telencephalic region is a striatum.
28. The method of embodiment 25-27, wherein the brain region within the telencephalic region is an extended amygdala.
29. The method of embodiment 25-28, wherein the brain region within the telencephalic region is an amygdala.
30. The method of embodiment 15-29, wherein the brain region is within a diencephalon region.
31. The method of embodiment 30, wherein the brain region within the diencephalon region is a posterior tuberculum.
32. The method of embodiment 30, wherein the brain region within the diencephalon region is a hypothalamus.
33. The method of any of embodiments 15-32, further including detecting activation of the portion of the brain region by imaging.

### (iv) Experimental Examples

Hedonic valence is a measurement of the intrinsic value of a stimulus, and can be positive (attractive), negative (aversive), or neutral. Pain typically has a negative valence, which is normally advantageous, as it drives self-protective behavior. In chronic pain conditions, however, this ordinarily helpful sense becomes maladaptive, and the negative valence associated with these disordered affective states can fuel suffering. Conversely, humans can sometimes assign a positive valence to nociceptive stimuli, for example finding pleasure in spicy foods. This implies that the neural circuits that assign negative valence to nociceptive stimuli are malleable, and that pain and aversion can be decoupled, providing a potential avenue for therapeutic intervention.

How motivational valence is assigned in the brain has been and continues to be the subject of much research and discussion (Tye, Neuron. 2018; 100:436-452; Namburi et al., Neuropsychopharmacology. 2016; 41:1697-1715; Namburi et al., Nature. 2015; 520:675-678; Berridge, Nat. Rev. Neurosci. 2019; 20:225-234). In mammals the determination of aversive motivational valence has been attributed to a number of areas within the central nervous system (CNS) most notably the striatum and the amygdala (Hikida et al., Neuron. 2010; 66; 896-907; Janak et al., Nature. 2015; 517: 284-292; Kim et al., Neuron. 2017; 201793:1464-1479.e5; Klawonn et al., Clin. Invest. 2018; 128: 3160-3170; Lerner et al., Cell. 2015; 162: 635-647; Paton et al., Nature. 2006; 439: 865-870; Pignatelli & Bonci, Neuron. 2015; 86:1145-1157; Wang et al., Nature. 2018; 558: 127-131). With respect to painful stimuli these areas as well as the anterior cingulate cortex (ACC), insula, the thalamus, habenula, hypothalamus, and brainstem nuclei including the parabrachial nuclei have also been implicated in assigning negative affect (Chiang et al., J. Neurosci. 2019; 39:8225-8230; Han et al., Cell. 2015; 162:363-374; Johansen et al., Nat. Neurosci. 2004; 7:398-403; Johansen et al., Proc. Natl. Acad. Sci. U.S.A 2001; 98: 8077-8082; Lazaridis et al., Mol. Psychiatry, 2019; 24:1351-1368; Lu et al., Neurosci. Bull. 2016; 32: 191-201; Shelton et al., Prog. Neurobiol. 2012; 96:208-219; Wang et al., Elife. 2017; 6:1-20: Zhang et al., Elife. 2017; 6:1-19; Price, Science. 2000; 288:1769-1772). Doparninergic signaling within the mesolimbic system has long been associated with reward or providing a positive valence for pleasurable stimuli. In the presence of noxious stimuli, the dopamine reward system is actively repressed, driving activation of circuits that promote aversion (Danjo et al., Proc. Natl. Acad. Sci. U.S.A 2014; 111: 6455-6460; McCutcheon et al., Front. Neurosci. 2012; 6: 1-10; Ungless et al., Science. 2004; 303: 2040-2042). Intriguingly, activation of dopamine signaling has also been shown to have anti-nociceptive effects and to participate in endogenous analgesic pathways, for example stress induced analgesia (Altier & Stewart. Life Sci. 1999; 65: 2269-2287; Gear et al., J. Neurosci. 1999; 19: 7175-7181; Puopolo. Neural Regen. Res. 2019; 14: 925-930; Schmidt et al., J. Neurosci. 2002:22:6773-6780). Despite great advances in understanding the neuronal circuits regulating pain sensation, however, there remain significant deficits in the understanding of how negative valence is attributed to noxious stimuli.

Zebrafish provide an attractive model system for inquiries into the biology of nociception. They can be generated in large numbers, have low maintenance costs, are easy to genetically manipulate, and their small size and optical clarity allow for large scale behavioral analysis and whole nervous system activity profiling. The organization of peripheral and central nociceptive processing systems is remarkably similar between teleost fish such as zebrafish and other vertebrates such as rodents and humans (Braithwaite et al., Dis. Aquat. Organ. 2007; 75:131-138; Prober et al., J. Neurosci. 2008:28:10102-10110; Sneddon, Neurosci. Lett. 2002; 319:167-171; Sneddon, Brain Res. 2003; 972:44-52). Even at timepoints as early AS1-3 days post fertilization (dpf), this nociceptive processing system is similarly organized and functional (Prober et al., J. Neurosci. 2008; 28:10102-10110; Caron et al., Development. 2008; 135:3259-3269; Curtright et al., PLoS One. 2015; 10:1-18; Esancy et al., Elife. 2018; 7:1-24; Gau et al., PLoS Genet. 2017; 13:1-30; Gau et al., Ann. Intern. Med. 2013; 158:5249-5260; Pan et al., Development. 2012; 139: 591-600). While still developing, larval zebrafish are fully functioning animals, which must hunt for prey and assign the appropriate valence to salient stimuli in order to survive. Notably, anatomical and functional dopamine signaling pathways are conserved in larval zebrafish and subcortical structures of the zebrafish telencephalon and diencephalon analogous to the striatum, amygdala, hypothalamus and habenula have been implicated in driving reward and aversion (Cheng et al., Sci. Rep. 2016:6:1-10: Cheng et al., Philos. Trans. R. Soc. B Biol. Sci. 2014; 369(1637):20120462; Filippi et al., J. Comp. Neurol. 2010; 518:423-438; Kastenhuber et al., J. Comp. Neurol. 2010; 518:439-458; Khan et al., Br. J. Pharmacol. 2017; 174:1925-1944; Krishnan et al., Curr. Biol. 2014; 24:1167-1175; Li et al., Dev. Dyn. 2007; 236:1339-1346; Reinig et al., Curr. Biol. 2017; 27:318-333; Souza et al., J. Neurosci. 2011; 31:5512-5525; Turner et al., Front. Neural Circuits. 2016; 10: 1-18; Lau et al., Proc. Natl. Acad. Sci. U.S.A 2011; 108:2581-2586; von Trotha et al., Eur. J. Neurosci. 2014; 40:3302-3315; Zhang et al., Neuron. 2017; 93:914-928.e4). These findings show that the neural circuits underpinning the determination of appetitive or aversive valence are largely conserved between larval zebrafish and mammals.

To investigate how valence is assigned to nociceptive stimuli, an operant place aversion assay in larval zebrafish was utilized to screen a small molecule library to identify compounds that alter aversion to noxious thermal stimuli. Herein, described is a small molecule, AS1, which remarkably reverses the valence of noxious stimuli, rendering them attractive. The effects of, AS1 were dose-dependent such that an intermediate dose could erase the aversion to the noxious stimulus without evoking preference. These results suggest that the setting of valence (appetitive, neutral or aversive) can be effectively tuned. Furthermore, AS1-induced attraction to noxious stimuli was directly proportional to the intensity of the noxious stimuli. Without being limited by mechanism or theory, the effects of AS1 were found to be dependent on DA signaling via D1 dopamine receptors, suggesting that AS1 may elicit its effects by relieving an intensity-encoded pain-imposed “brake” on DA release. This is in contrast to addictive opioid analgesics such as morphine, which activate reward circuitry independent of context while simultaneously suppressing nociceptive circuitry.

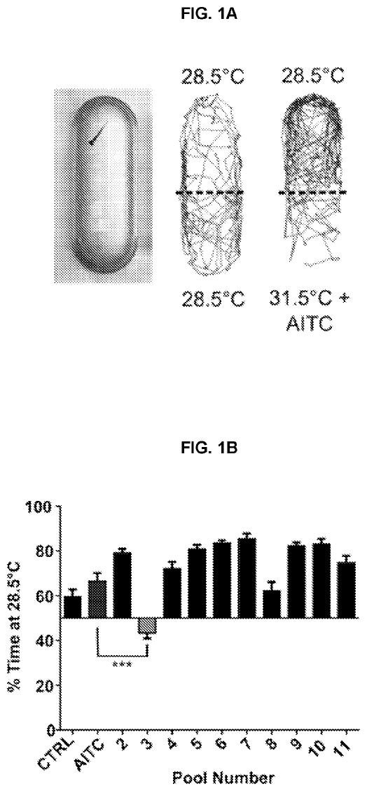

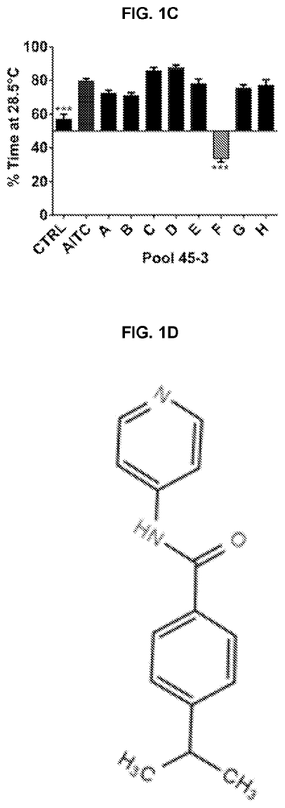

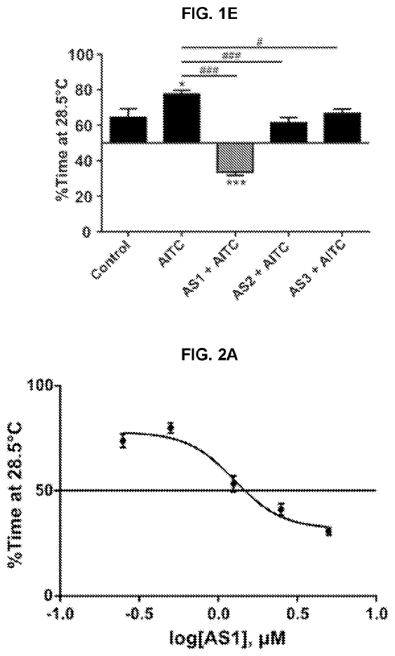

The Novel Analgesic AS1 reverses the valence of normally aversive stimuli. In a previously published study, a novel high throughput temperature discrimination assay utilizing larval zebrafish that modeled acute and sensitized temperature aversion were developed (FIG. 1A) (Curtright et al., PLoS One. 2015; 10:1-18). The assay revealed that 5 dpf zebrafish are exquisitely averse to temperatures that deviate from rearing temperature (28.5° C.), in a temperature dependent manner. In the sensitized temperature aversion assay, larval zebrafish were pre-incubated in the inflammatory noxious chemical irritant allyl isothiocyanate (AITC) at a concentration that does not evoke locomotor escape behaviors (0.5 μM). When given a choice between their rearing temperature (28.5° C.) and mildly aversive heat (31.5° C.), larvae pre-incubated in this sub-threshold AITC concentration demonstrated greatly potentiated aversion to the 31.5° C. zone, mimicking conditions of thermal hyperalgesia. Also shown is that multiple small molecule analgesics could reverse acute and/or sensitized thermal aversion while non-analgesics could not. These studies suggest that thermal aversion is reflective of nociceptive behavior and that the assays could be an important tool in the identification of small molecules that disrupt or enhance nociceptive behavior and their cellular and molecular targets (Curtright et al., PLoS One. 2015; 10:1-18).

Using the sensitized thermal aversion assay, a small molecule library was screened in order to identify targets in pain transduction pathways and potential entry points for therapeutic intervention. The compounds were selected from Chembridge's CNS-Set (https://www.chembridge.com), which consists of small molecules selected for blood brain barrier penetration and oral bioavailability. Compounds were selected from across the library to maximize the diversity of molecules screened. Small molecules were initially pooled (8 per pool) to maximize screening efficiency. Larvae (n of 64) in individual choice testing arenas were incubated in small molecule pools for 10 min followed by the addition of the noxious transient receptor potential cation channel subfamily A member 1 (TRPA1) agonist, allyl isothiocyanate (AITC) (0.5 μM), and then tested for thermal preference (28.5° vs 31.5° C.) (Prober et al., J. Neurosci. 2008:28:10102-10110; Jordt et al., Nature. 2004; 427:260-265; Bautista et al., Cell. 2006; 124:1269-1282). A pool from plate 45, column 3 that remarkably appeared to induce a slight preference to the noxious stimuli was identified (FIG. 1B). Demultiplexing this pool identified one compound (4-propan-2-yl-N-pyridin-4-ylbenzamide, hereafter referred to as AS1), with no known prior biological activity, that induced place preference for the noxious zone (FIG. 1B). In addition to AS1, two other molecules (AS2 and AS3) that blocked the sensitized thermal aversion elicited by AITC were identified (FIG. 1B). Notably, these two molecules, like previously tested analgesic compounds, only lessened aversion for the noxious zone, dramatically differing from the AS1-induced preference for noxious zone that was observed.

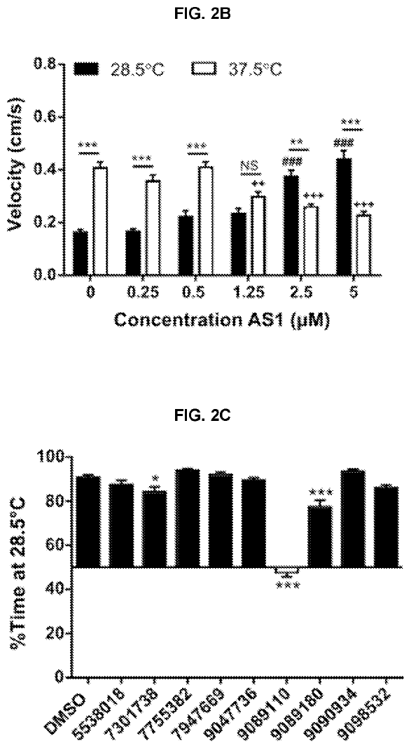

To determine if the effects of AS1 extended to acute noxious temperature, dose-response analysis was performed to determine the effects of AS1 on acute heat aversion (28.5° vs 37.5° C.). The highest dose of AS1 (5 μM) evoked strong preference for the nociceptive stimulus. At lower doses, AS1 had intermediate effects, either reducing aversion or inducing a neutral response where the noxious stimulus was neither aversive nor attractive (FIG. 2). Notably, AS1 also inverted zone-dependent locomotor activity when compared to vehicle-treated larvae, suggesting that the 28.5° C. zone became aversive in addition to the 36.5° C. zone becoming attractive (FIG. 2). This demonstrates that the setting of valence (appetitive, neutral or aversive) can be effectively tuned, and that AS1 may be acting upon a specific target.

To potentially identify the specific aspect of AS1's structure that mediates its effects, the thermal aversion assay was performed with structurally similar chemical analogs of AS1 (FIG. 2, Table 1). Of the nine chemicals that were tested, three were found that significantly altered the amount of time spent in the 28.5° C. zone, and only one of which, Analog 9089110 (4-propyl-N-4-pyridinylbenzamide), completely ablated aversion in a similar manner as AS1, although its effects were less potent and it did not induce a strong preference for the 37.5° C. zone at the tested concentration. The only structural difference between AS1 and Analog 9089110 is that the latter has a propyl group, as opposed to a propan-2-yl group, emerging from the benzene ring of the N-pyridin-4-ylbenzamide backbone that both of these molecules share. Without being limited by mechanism or theory, this suggests that while this particular functional group might be dispensable for AS1's action, overall the rest of the chemical structure may be important to elicit AS1's molecular effects. For example, Analog 7301738 (4-isopropyl-N-3-pyridinylbenzamide) differs from AS1 only in the position of the nitrogen atom in the pyridine ring, yet it only had a small, if still significant, effect on temperature preference (FIG. 2). The possible importance of this nitrogen atom may be underscored by the fact that Analog 9090934 (4-propyl-N-3-pyridinylbenzamide), which is identical to Analog 9089110 except for the position of this atom, similarly has no effect upon temperature preference. Lengthening the alkyl group attached to the benzamide ring also prevented an analogue from replicating the effects of AS1 (as with Analog 9098532, or 4-butyl-N-4-pyridinylbenzamide), as did modifying the position and number of alkyl groups attached to the benzamide (Analogs 7755382 and 9047736, 2,4-dimethyl-N-4-pyridinylbenzamide and 2,4,6-trimethyl-N-4-pyridinylbenzamide, respectively). Intriguingly, the only other molecule that had a significant (if small) influence upon temperature choice behavior (Analog 9089180, or N-(4-isopropylphenyl)-3,5-dimethyl-4-isoxazolecarboxamide) exhibited the greatest level of structural difference from AS1 out of all of the molecules that were tested. It is possible that this chemical is eliciting mild analgesia via a different mechanism than that of AS1.

TABLE 1
Structural Analogs of AS1 and Their Effects Upon Temperature Preference

| | | | Reverses |
|---|---|---|---|
| Compound | | | 28.5° C. |
| # | Name | Structure | Choice? |
| 5538018 | N-(4- isopropylphenyl)nicotinamide | 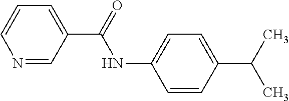 [SMILES: CC(C)c1ccc(NC(=O)c2cccnc2)cc1] | No |
| 7301738 | 4-isopropyl-N-3- pyridinylbenzamide | 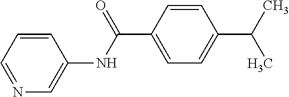 [SMILES: CC(C)c1ccc(C(=O)Nc2cccnc2)cc1] | No |
| 7755382 | 2,4-dimethyl-N-4- pyridinylbenzamide | 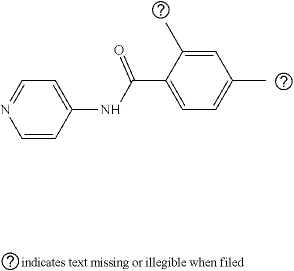 [SMILES: Cc1ccc(C(=O)Nc2ccncc2)c(C)c1] | No |
| 7947669 | 4-ethyl-N-3- pyridinylbenzamide | 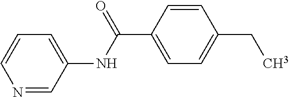 [SMILES: CCc1ccc(C(=O)Nc2cccnc2)cc1] | No |
| 9047736 | 2,4,6-trimethyl-N-4- pyridinylbenzamide | 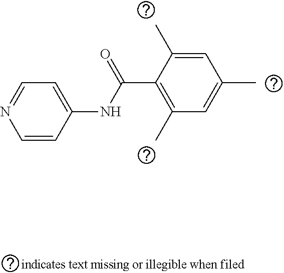 [SMILES: Cc1cc(C)c(C(=O)Nc2ccncc2)c(C)c1] | No |
| 9089110 | 4-propyl-N-4- pyridinylbenzamide | 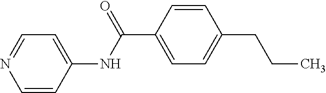 [SMILES: CCCc1ccc(C(=O)Nc2ccncc2)cc1] | No preference for either temperature |
| 9089180 | N-(4-isopropylphenyl)-3,5- dimethyl-4- isoxazolecarboxamide | 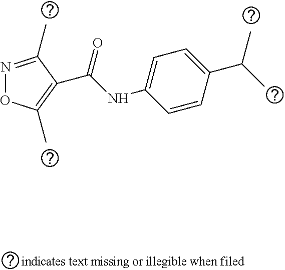 [SMILES: Cc1noc(C)c1C(=O)Nc1ccc(C(C)C)cc1] | No |
| 9090934 | 4-propyl-N-3- pyridinylbenzamide | 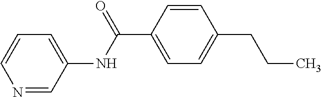 [SMILES: CCCc1ccc(C(=O)Nc2cccnc2)cc1] | No |
| 9098532 | 4-butyl-N-4- pyridinylbenzamide | 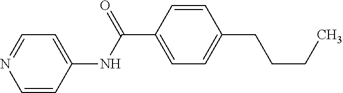 [SMILES: CCCCc1ccc(C(=O)Nc2ccncc2)cc1] | No |
| 7264699 | 4-propan-2-yl-N-pyridin-4- ylbenzamide (AS1) | 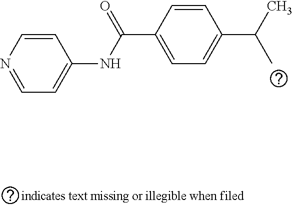 [SMILES: CC(C)c1ccc(C(=O)Nc2ccncc2)cc1] | Yes |

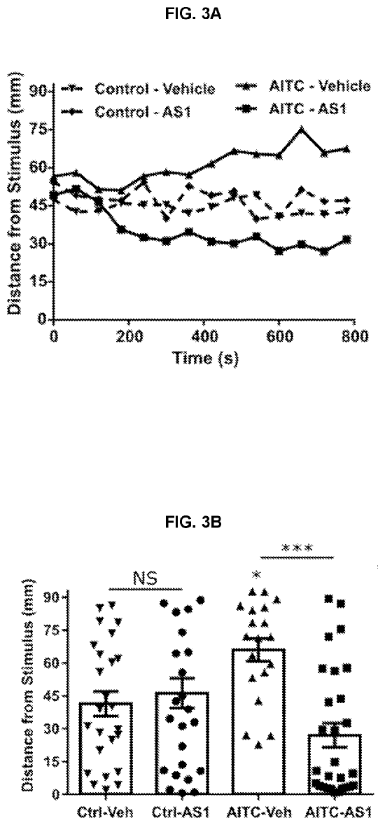

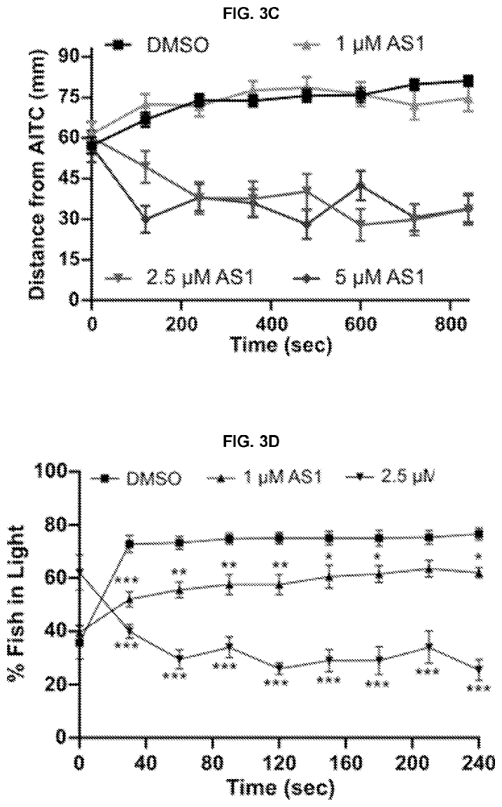

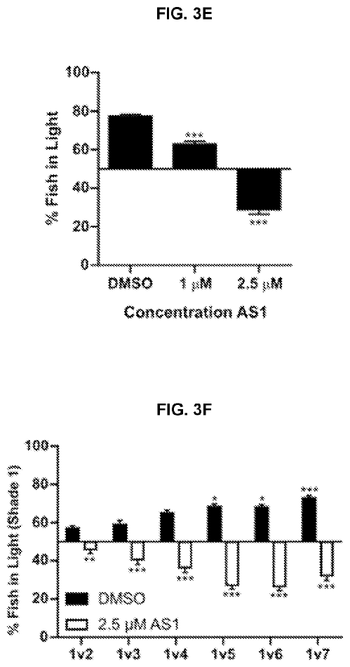

Whether the effects of AS1 extended beyond noxious temperature sensation or could also impact other somatosensory modalities was next explored. To accomplish this, a chemical attraction/aversion assay where a thin layer of agarose was deposited along the edges of a square arena (Jordi et al., Sci. Adv. 2018; 4:1-15) were adapted. Along one wall, the agarose contained the chemical to be tested, while the other three walls were lined with control agarose. It was anticipated that a chemical gradient would be established as chemical diffused from the agarose into the surrounding water. Larvae were pre-incubated in either vehicle or AS1 and then added to the arena and the distance of each larva from the chemical-infused agarose was measured. In control experiments where all four walls of the arena were lined with agarose only, AS1 and vehicle-treated larvae were dispersed evenly throughout the arena (FIG. 3). This demonstrated that neither the arena nor AS1 itself induced preference/aversion for any side of the arena. When the nociceptive chemical AITC (100 mM, 10,000× maximal effective dose) was tested, vehicle-treated larvae swam away from the AITC source. Remarkably, AS1-treated larvae strongly preferred to be near the AITC source, with the majority of larvae swimming directly to the AITC source, indicating attraction to this potently noxious nociceptive stimulus (FIG. 3) Performing dose-response analysis revealed that 1 μM AS1 may be unable to induce attraction to AITC whereas 2.5 μM AS1 largely replicated the effects of 5 μM AS1 (FIG. 3).

Following this, whether the effects of AS1 were restricted to somatosensation or were generalizable to other aversive stimuli were tested. Thus, the effects of AS1 on light/dark preference were tested. Zebrafish larvae prefer white light environments to dark environments and this preference is inhibited by anxiolytics, analagous to studies in rodents (Lau et al., Proc. Natl. Acad. Sci. U.S.A 2011; 108:2581-2586; Burgess et al., Curr. Biol. 2010; 20:381-386). To measure light/dark preference, larval zebrafish were placed in a square arena and given the choice between a bright white light and total darkness, and the number of larvae on each side was quantified at 30 second intervals. Five 4-minute trials were performed, where the light and dark sides of the arena were reversed between trials. In alignment with previous findings using heat and chemical stimuli, AS1 reversed light/dark preference in a dose-dependent manner, with AS1-treated larvae strongly preferring the dark environment whilst vehicle-treated fish preferred the light side of the chamber (FIG. 3). Given the precise control over stimulus luminosity in this assay, the effects of altering the intensity of the dark stimulus were explored next. In this gradient version of the phototaxis assay, larval zebrafish were given the choice between a bright white environment and one of six potential shades of darkness, ranging from light gray to solid black. Interestingly, the effects of AS1 scaled with the intensity of the dark stimulus in a way inverse to the observation with control fish—while darker and darker shades of gray elicited greater and greater avoidance of the dark half of the arena in DMSO-treated fish, AS1-treated fish demonstrated greater and greater attraction to the dark (FIG. 3). This implies that the valence of non-painful noxious stimuli is precisely encoded in an intensity-dependent manner, and that AS1 exerts its effects in proportion to the intensity of the stimulus. Collectively, these data show that AS1 can ablate aversion and instead induce preference for both nociceptive and other aversive stimuli.

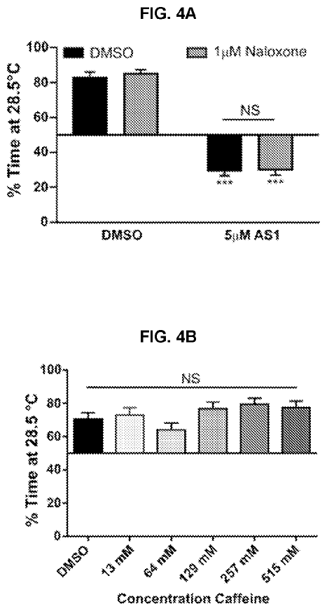

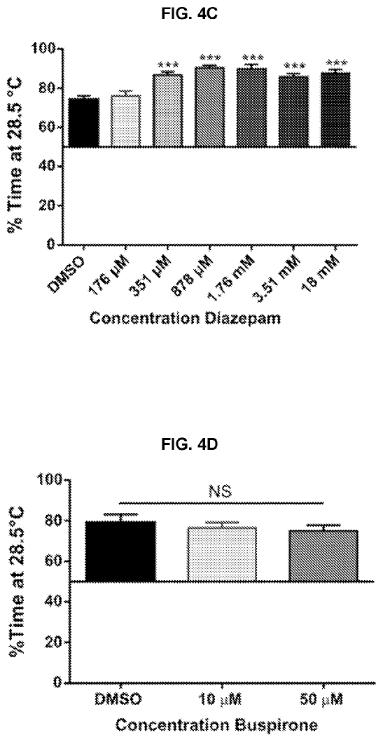

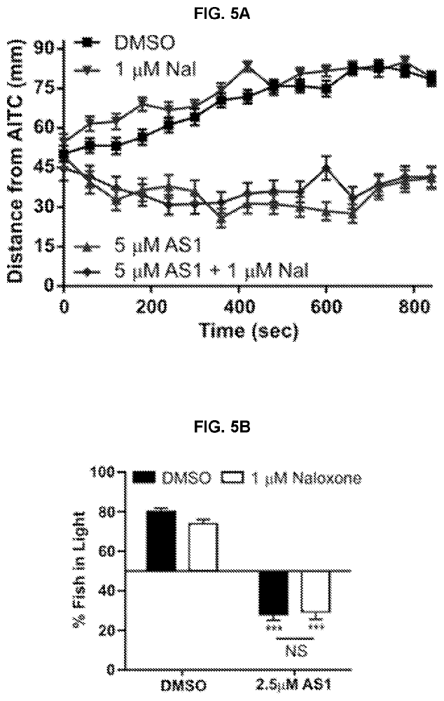

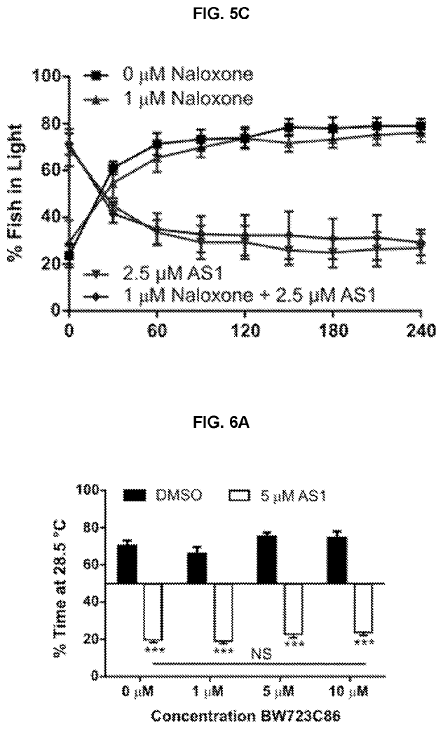

Canonical pain-relief circuitry is not involved in mediating the effects of AS1. Once it had been established that AS1 could ablate aversion and induce preference for aversive stimuli across sensory modalities, the neural mechanisms underlying these effects upon hedonic valence were studied. Given the wealth of literature upon opioid analgesics-which have antinociceptive properties as well as engage reward/valence circuitry—the possibility of whether AS1 might be acting in a similar fashion was explored. Both in vitro and in vivo studies suggest that zebrafish mu opioid receptor (MOR) has a pharmacological profile similar to that of mammalian MORs, and that its activation elicits analogous physiological and behavioral effects (De Velasco et al., Zebrafish. 2009; 6(3):259-268; Sivalingam et al., Front Neuroanat. 2020; 14:5; Costa et al., Neurosci. Lett. 2019; 708: 134336; Zaig et al., Elife. 2021; 10:1-20). Naloxone, a MOR antagonist, did not replicate or attenuate the effects of AS1 in the thermal preference, AITC aversion, or phototaxis assays (FIG. 4, FIG. 5).

Also tested was whether treatment with stimulants or anxiolytics could replicate the effects of AS1 in the thermal aversion assay. Some anxiolytics have been shown to have analgesic properties, and in zebrafish, can also attenuate light preference (Lau et al., Proc. Natl. Acad. Sci. U.S.A 2011; 108:2581-2586; Lax et al., PLoS One. 2014; 9(7):e103524). Likewise, many stimulants have intrinsic analgesic properties, can potentiate the effects of opioid analgesia, and have been used by people as a self-medication strategy to treat chronic pain (Dalal & Melzack, J. Pain Symptom Manage. 1998; 16: 245-253; Opperman et al., Prim Care Companion CNS Disord. 2021; 23(1):20102620). Treatment with the stimulant/anxiogenic caffeine did not affect thermal preference for 28.5° C. at any of the concentrations that were tested (FIG. 4). Similarly, treatment with the anxiolytic drugs that were tested (diazepam and buspirone) did not elicit attraction to lethal heat at any tested concentration. In fact, some concentrations of diazepam potentiated preference for 28.5° C. (FIG. 4). Buspirone by contrast had no effect upon thermal preference (FIG. 4).

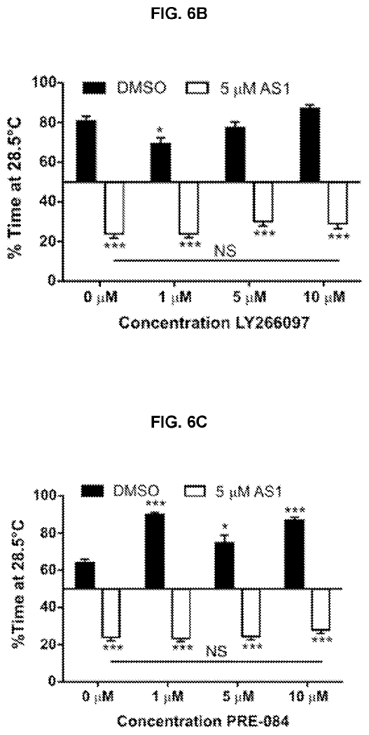

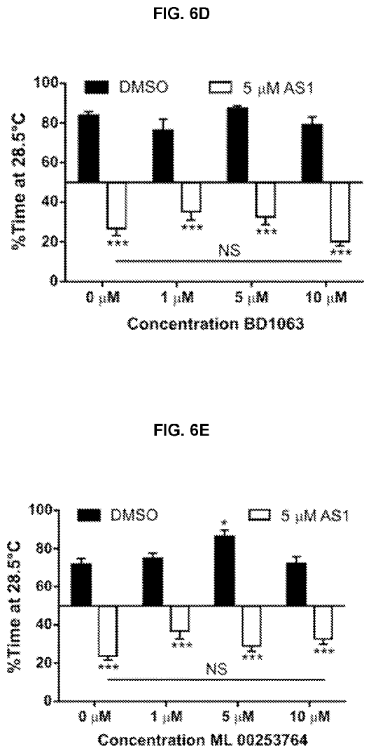

To further seek out the molecular target(s) of AS1, the resources of the Psychoactive Drug Screening Program (PDSP) (Besnard et al., Nature. 2012; 492:215-220) were utilized. Radioligand binding assays identified two receptors that had potentially weak interactions with AS1, the 5-HT₂a serotonin receptor and the sigma-1 receptor. While not being limited to mechanism or theory, to verify whether these receptors were involved in mediating the effects of AS1, the thermal preference assay following incubation in drugs that specifically acted upon these receptors was employed. Treatment with the 5-HT₂₉ agonist BW723C86 did not appear to replicate or reverse the effects of AS1 (FIG. 6). Nor did treatment with the 5-HT₂ s antagonist LY266097 replicate or reverse the effects of AS1 (FIG. 6). Incubation in either the specific sigma-1 receptor agonist (PRE-084) or antagonist (BD1063) similarly yielded no effects (FIG. 6). These data suggest that AS1 may not be acting on either of these receptors to mediate its effects.

Whether the melanocortin 4 receptor (MCR4), which has previously been implicated in valence reversal of nociceptive stimuli in rodents, could underlie the effects of AS1 (Klawonn et al., Clin. Invest. 2018; 128: 3160-3170; DiFeliceantonio et al., J. Clin. Invest. 2018; 128: 2757-2759) was tested. While not being limited to mechanism or theory, treatment with the MCR4 antagonist ML00253764 likewise did not replicate or attenuate AS1-induced attraction to noxious heat at any of the concentrations that were tested (FIG. 6).

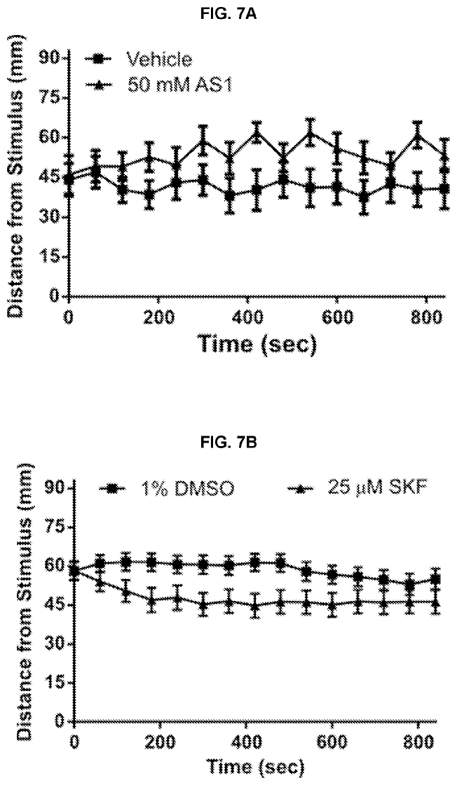

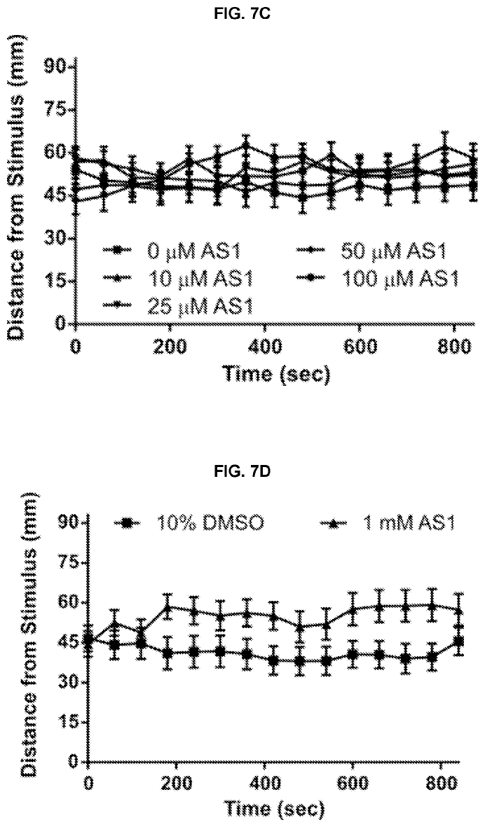

To confirm that AS1 itself was not attractive, the chemical attraction/aversion assay was used. When agarose containing AS1 at a concentration of 50 mM (10,000× the effective dose) was deposited against one side of a square arena, larval zebrafish did not approach it in a way that was appreciably different from control agarose: if anything, AS1 appeared to be slightly aversive (FIG. 7). By contrast, the D1 dopamine receptor agonist SKF82958 (selected with the expectation that activating D1 circuitry should be rewarding) did appear to be mildly attractive at various concentrations (FIG. 7). Since the assay was modified to accommodate the inability to dissolve SKF into agarose at high concentrations (see Materials and Methods), the inherent attractiveness of AS1 was re-tested using this new format. As expected based upon the initial results, AS1 continued to be neutral or mildly aversive for larval zebrafish even at higher concentrations (FIG. 7).

Without being limited by mechanism or theory, together, this data suggests that AS1 may be acting to reverse valence via a unique molecular mechanism unlike those underlying traditional analgesics. Additionally, it does not possess any intrinsic attractiveness that have been observed in other drugs, such as opioid analgesics (McKendrick et al., Front. Behav. Neurosci. 2020; 14: 1-15; Rossi et al., Physiol. Psychol. 1976; 4:269-274; Katz & Gormezano, Pharmacol. Biochem. Behav. 1979; 11: 231-233). Instead, AS1 appears to elicit attraction only in the presence of a noxious stimulus, suggesting that activation of aversion-encoding neural circuitry is required for the observed hedonic shift.

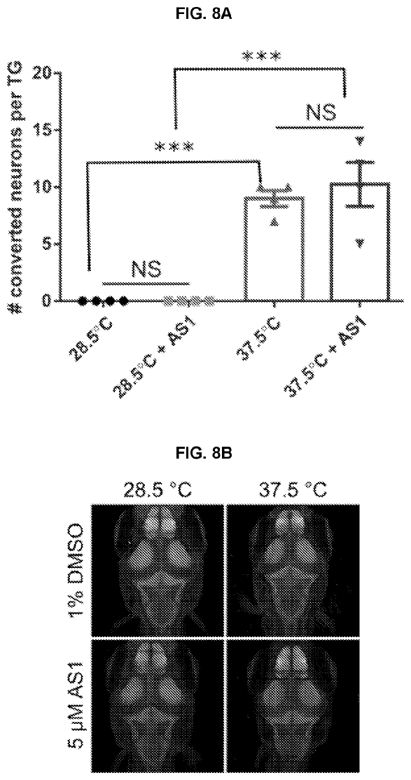

Brain regions associated with dopaminergic circuitry are specifically activated in the concurrent presence of AS1 and noxious stimuli. It was next sought to determine in an unbiased manner where in the zebrafish nervous system AS1 was exerting its effects by examining neuronal activity in the context of noxious stimuli. While the ability of AS1 to modify the valence of aversive stimuli across multiple sensory modalities suggested that it may have acted via central nervous system mechanisms, the data did not rule out the possibility that peripheral nervous system mechanisms were also involved. Many analgesics can act upon multiple different levels of pain transduction circuitry. For example, MORs can be found upon peripheral somatosensory neurons, spinal cord neurons, and numerous neuronal populations in the brain, and both exogenous and endogenous opioids can modulate the activity of any of these neurons (Corder et al., Annu. Rev. Neurosci. 2018; 41: 453-473). To investigate whether peripheral somatosensory neurons were also influenced by AS1, a neuronal activity assay upon transgenic zebrafish expressing the genetically-encoded calcium indicator CaMPARI in all neurons was performed. This fluorescent protein permanently photoconverts from green to red in the presence of a 405 nm light and high calcium (a proxy for neuronal activity), allowing “snapshots” of neuronal activity at a single time point (Fosque et al., Science. 2015; 347(6223):755-760). Fish were exposed to conditions of 28.5° C. or 37.5° C. with or without AS1 in the presence of a blue light, and then the trigeminal ganglia (TG) were surveyed for photoconverted neurons. As expected, exposure to the rearing temperature of 28.5° C. did not elicit any conversion of trigeminal neurons in control zebrafish, whereas exposure to 37.5° C. led to the photoconversion of significantly more neurons (FIG. 8). AS1-treated fish likewise exhibited an absence of TG neuronal activity under conditions of 28.5° C. and robust activity under exposure to noxious heat (FIG. 8). No significant difference between AS1-treated and control fish was observed at either temperature. Without being limited by mechanism or theory, this data suggests that AS1 is not directly modulating the activity of peripheral heat sensitive somatosensory neurons either by itself or in the presence of a nociceptive stimulus, and suggests that AS1 is likely acting downstream of these neurons.

To investigate how AS1 might alter central nervous system activity in the presence of nociceptive stimuli, whole brain activity profiling with the neuronal activity marker phosphorylated-ERK (pERK) was performed. Briefly, 6 dpf larval zebrafish were exposed to noxious heat (37.5° C.) or rearing temperature (28.5° C.) in the presence of vehicle or AS1 (either 2.5 or 5 μM) for 15 minutes. Immunolabeling was then performed to detect both total ERK (tERK) and phosphorylated ERK (pERK) (FIG. 8). Volumetric z-stacks of the entire brain of each fish were taken upon a confocal microscope, registered to a reference brain, and smoothed using an ImageJ script. A previously established pipeline was then used to quantify how neuronal activity was up- or down-regulated in each annotated brain region in the zebrafish CNS between different groups of fish (Randlett et al., Nat. Methods. 2015; 12:1039-1046). Brains from each experimental condition (AS1+28.5° C., Vehicle+37.5° C., and AS1+37.5° C.) with Vehicle+28.5° C. were initially compared to determine how activity under these experimental conditions differed from the baseline state, then the total change in signal in each brain region were subtracted for AS1+28.5° C. and Vehicle+37.5° C. treated fish from AS1+37.5° C. treated fish. This was done to look for brain regions specifically activated in the presence of the noxious stimulus and AS1, reasoning that these areas would drive attraction to noxious stimuli.

Strikingly, in the presence of noxious heat and AS1, a large proportion of the most highly activated regions were located in the zebrafish subpallium, a broad telencephalic region that has been described as the equivalent of the mammalian basal ganglia, striatum, and extended amygdala (Table 2) (Porter & Mueller, Front Neurosci. 2020; 14:608; Ganz et al., J. Comp. Neurol. 2012; 520:633-655; Mueller et al., J. Comp. Neurol. 2008; 507:1245-1257). Dopaminergic regions, both within the subpallium and in the diencephalon (e.g., posterior tuberculum, hypothalamus), were also heavily represented (Table 2). Additionally, diencephalic neuronal clusters classified by expression of genes required for dopaminergic development/that also label dopaminergic neurons, such as Otpb and Isl1, were also active to a high degree (Table 2). Intriguingly, AS1 does not appear to indiscriminately activate dopaminergic subpopulations-instead, only certain clusters appear to be recruited by the tandem application of AS1 and heat. Together, these populations include 64% of the top 25 most active regions. Other highly active regions included oxytocin (OXTL) neuronal clusters, which play roles in stress relief and nociception; telencephalic white matter tracts, other basal ganglia precursors such as the thalamic eminence, and hypocretin (Hcrt) and pyroglutamylated RFamide peptide (Qrfp) clusters, which are involved in arousal and motivation. Interestingly, in AS1-treated fish that were not exposed to noxious heat, most of these regions were not highly active-rather, AS1 alone primarily recruited neuron clusters within the mesencephalon and rhombencephalon (midbrain/hindbrain), although some diencephalic OXTL, Hcrt, and Qrfp clusters are still represented.

TABLE 2
Brain Regions Specifically Upregulated in the Context of 5 μM AS1 and 37.5° C.

| | Signal Specific to |
|---|---|
| ROI | AS1 + Heat |
| Telencephalon - Isl1 cluster 1 | 62784.02 |
| Telencephalon - Isl1 cluster 2 | 59449.99 |
| Telencephalon - Subpallial Otpb Cluster 2 | 57862.31 |
| Telencephalon - S1181t Cluster | 55333.89 |
| Diencephalon - Eminentia Thalami | 55198.24 |
| Telencephalon - Anterior Commisure | 52049.98 |
| Telencephalon - Subpallial Gad1b cluster | 51995.37 |
| Telencephalon - Olfactory bulb dopaminergic neuron areas | 51323.12 |
| Telencephalon - Subpallium | 50437.3 |
| Diencephalon - Otpb Cluster 2 | 48466.37 |
| Diencephalon - Retinal Arborization Field 4 (AF4) | 47600.85 |
| Diencephalon - Oxtl Cluster 1 in Preoptic Area | 47413.8 |
| Diencephalon - Retinal Arborization Field 2 (AF2- Approximate Location) | 46758.78 |
| Diencephalon - Preoptic area Vglut2 cluster | 46521.07 |
| Telencephalon - Vmat2 cluster | 46517.43 |
| Telencephalon - Subpallial Otpb strip | 45224.41 |
| Telencephalon - Subpallial dopaminergic cluster | 44557.21 |
| Diencephalon - Retinal Arborization Field 3 (AF3) | 43716.94 |
| Diencephalon - Dopaminergic Cluster 3 - hypothalamus | 40019.12 |
| Diencephalon - Isl1 cluster 1 | 39266.09 |
| Telencephalon - | 38670.72 |
| Rhombencephalon - Glyt2 Cluster 8 | 38553.58 |
| Telencephalon - Subpallial Vglut2 Cluster | 38145.97 |
| Diencephalon - Isl1 cluster 2 | 36475.6 |
| Telencephalon - Pallium | 36091.17 |
| Diencephalon - Otpb Cluster 4 | 35964.5 |
| Diencephalon - Hypothalamus Qrfp neuron cluster | 35847.32 |
| Rhombencephalon - Olig2 Cluster | 35771.76 |
| Rhombencephalon - Gad1b Cluster 8 | 35509.66 |
| Diencephalon - Hypothalamus Hort Neurons | 35066.89 |
| Telencephalon - Telencephalic Migrated Area 4 (M4) | 35038.42 |
| Diencephalon - Oxtl Cluster 5 | 34474.08 |
| Diencephalon - Left Habenula Vglut2 Cluster | 33383.12 |
| Rhombencephalon - Glyt2 Cluster 7 | 33307.91 |
| Diencephalon - Oxtl Cluster 2 | 32549.34 |
| Rhombencephalon - MiM1 | 32356.03 |
| Diencephalon - Dopaminergic Cluster 1 - ventral thalamic and | 31653.23 |
| periventricular posterior tubercular DA neurons | |
| Diencephalon - Hypothalamus s1181t Cluster | 30757.2 |
| Telencephalon - Vglut2 rind | 30426.53 |
| Mesencephalon - Vmat2 cluster2 | 28483.57 |
| Diencephalon - Olig2 Band 2 | 27055.59 |
| Rhombencephalon - Spiral Fiber Neuron Posterior cluster | 26252.82 |
| Rhombencephalon - Mauthner | 26157.52 |
| Diencephalon - Preoptic Area | 26037.78 |
| Rhombencephalon - Gad1b Cluster 6 | 25823.45 |
| Telencephalon - Olig2 Cluster | 25415.34 |
| Telencephalon - Olfactory Bulb | 24123.37 |
| Diencephalon - Otpb Cluster 3 | 23189.83 |
| Rhombencephalon - Vglut2 cluster 1 | 23112.53 |
| Diencephalon - Ventral Thalamus | 22613.96 |
| Rhombencephalon - Otpb Cluster 1 | 22484.27 |
| Diencephalon - Hypothalamus Gad1b Cluster 2 | 22390.58 |
| Diencephalon - Hypothalamus Vglut2 Cluster 2 | 21908.39 |
| Rhombencephalon - Otpb Cluster 5 | 21365.88 |
| Rhombencephalon - Isl1 Cluster 2 | 20801.39 |
| Diencephalon - Preoptic Otpb Cluster | 19626.58 |
| Rhombencephalon - Anterior Cluster of nV Trigeminal Motorneurons | 18215.71 |
| Diencephalon - Anterior group of the posterior tubercular vmat2 neurons | 18048.81 |
| Rhombencephalon - Gad1b Cluster 9 | 17895.14 |
| Rhombencephalon - Gad1b Cluster 18 | 17734.97 |
| Diencephalon - Migrated Posterior Tubercular Area (M2) | 17248.35 |
| Diencephalon - Right Habenula Vglut2 Cluster | 17020.66 |
| Diencephalon - Posterior Tuberculum | 16128.36 |
| Diencephalon - Dorsal Thalamus | 16051.33 |
| Diencephalon - Dopaminergic Cluster 4/5 - posterior tuberculum and | 15771.94 |
| hypothalamus | |
| Rhombencephalon - Otpb Cluster 2 - locus coeruleus | 15356.7 |
| Diencephalon - Preoptic area posterior dopaminergic cluster | 15298.49 |
| Rhombencephalon - RoL2 | 14726.04 |
| Diencephalon - Otpb Cluster 1 | 14710.13 |
| Diencephalon - Rostral Hypothalamus | 14599.99 |
| Rhombencephalon - Oxtl Cluster 2 Near MC axon cap | 14593.89 |
| Diencephalon - Habenula | 14090.36 |
| Diencephalon - Pretectal Gad1b Cluster | 13821.77 |
| Ganglia - Lateral Line Neuromast OC1 | 13662.37 |
| Rhombencephalon - Locus Coreuleus | 13413.93 |
| Rhombencephalon - Gad1b Cluster 19 | 12861.25 |
| Rhombencephalon - Spiral Fiber Neuron Anterior cluster | 11867.7 |
| Diencephalon - Retinal Arborization Field 1 (AF1- Approximate Location) | 11327.06 |
| Rhombencephalon - RoL3 | 11223.15 |
| Diencephalon - Pretectal dopaminergic cluster | 10597.32 |
| Mesencephalon - Vmat2 cluster of paraventricular organ | 10260.77 |
| Rhombencephalon - Ptf1a Cluster 1 | 9981.472 |
| Rhombencephalon - MiD2 | 9951.084 |
| Rhombencephalon - Gad1b Cluster 16 | 9758.203 |
| Diencephalon - Dopaminergic Cluster 2 - posterior tuberculum | 9182.1 |
| Diencephalon - | 8916.113 |
| Rhombencephalon - Posterior Cluster of nV Trigeminal Motorneurons | 8813.364 |
| Diencephalon - Hypothalamus Gad1b Cluster 1 | 8810.209 |
| Rhombencephalon - Cerebellum Gad1b Enriched Areas | 8478.309 |
| Rhombencephalon - RoM2 | 7858.519 |
| Rhombencephalon - Medial Vestibular Nucleus | 7788.739 |
| Rhombencephalon - Glyt2 Cluster 14 | 7646.447 |
| Rhombencephalon - RoM3 | 7221.729 |
| Diencephalon - Hypothalamus 6.7FRhcrtR-Gal4 cluster 1 | 6882.392 |
| Mesencephalon - Isl1 cluster of the mesencephalic region | 6687.923 |
| Rhombencephalon - ROL-R1 | 6683.713 |
| Diencephalon - Postoptic Commissure | 6193.115 |
| Rhombencephalon - Olig2 enriched areas in cerebellum | 6184.994 |
| Rhombencephalon - 6.7FDhcrtR-Gal4 Cluster 5 | 6097.938 |
| Telencephalon - Postoptic Commissure | 5446.596 |
| Diencephalon - Medial vglut2 cluster | 5322.251 |
| Rhombencephalon - Rhombomere 4 | 4929.172 |
| Diencephalon - Anterior pretectum cluster of vmat2 Neurons | 4910.732 |
| Rhombencephalon - Rhombomere 2 | 4539.056 |
| Rhombencephalon - Gad1b Cluster 5 | 4480.095 |
| Rhombencephalon - Rhombomere 3 | 4451.223 |
| Rhombencephalon - MiT | 4354.045 |
| Rhombencephalon - Glyt2 Cluster 3 | 4302.117 |
| Rhombencephalon - Glyt2 Cluster 10 | 4231.226 |
| Rhombencephalon - Vglut2 cluster 2 | 3738.04 |
| Diencephalon - Isl1 cluster 3 | 3443.261 |
| Rhombencephalon - Glyt2 Cluster 12 | 3375.659 |
| Rhombencephalon - Neuropil Region 4 | 3362.248 |
| Rhombencephalon - Otpb Cluster 3 | 2806.197 |
| Rhombencephalon - MiR1 | 2703.727 |
| Rhombencephalon - Glyt2 Cluster 5 | 2156.549 |
| Rhombencephalon - Vmat2 Cluster 1 | 2015.248 |
| Diencephalon - Intermediate Hypothalamus | 1944.787 |
| Rhombencephalon - Glyt2 Cluster 6 | 1824.908 |
| Diencephalon - Oxtl Cluster 4 - sparse in hypothalamus | 1645.072 |
| Rhombencephalon - MiV1 | 1292.555 |
| Rhombencephalon - Glyt2 Cluster 11 | 1206.636 |
| Rhombencephalon - Spinal Backfill Vestibular Population | 1168.953 |
| Rhombencephalon - Glyt2 Stripe 3 | 520.9109 |
| Rhombencephalon - 6.7FDhcrtR-Gal4 Cluster 2 Sparse | 421.7474 |
| Rhombencephalon - Rhombomere 5 | 347.6554 |
| Rhombencephalon - S1181t Cluster | 257.5919 |
| Diencephalon - Oxtl Cluster 3 | 230.0603 |
| Rhombencephalon - Gad1b Cluster 17 | 197.6007 |
| Diencephalon - Pineal | 166.653 |
| Diencephalon - Pineal Vmat2 cluster | 114.2982 |
| Rhombencephalon - Otpb Cluster 4 | 81.5449 |
| Ganglia - Statoacoustic Ganglion | 73.701 |
| Rhombencephalon - Mauthner Cell Axon Cap | 49.26 |
| Rhombencephalon - Isl1 Cluster 1 | 24.4084 |
| Rhombencephalon - Glyt2 Cluster 1 | 10.4124 |
| Mesencephalon - Torus Longitudinalis | −10.4916 |
| Rhombencephalon - Gad1b Cluster 13 | −367.094 |
| Rhombencephalon - Vmat2 Stripe3 | −433.666 |
| Rhombencephalon - Interpeduncular Nucleus | −569.553 |
| Rhombencephalon - Neuropil Region 6 | −581.833 |
| Rhombencephalon - Tangential Vestibular Nucleus | −582.271 |
| Rhombencephalon - Cerebellum | −619.091 |
| Rhombencephalon - Rhombomere 1 | −624.385 |
| Rhombencephalon - Corpus Cerebelli | −745.879 |
| Rhombencephalon - Isl1 Cluster 3 | −774.533 |
| Rhombencephalon - Vglut2 Stripe 3 | −774.994 |
| Rhombencephalon - Glyt2 Cluster 1 | −919.867 |
| Rhombencephalon - Vmat2 Cluster 5 | −1421.05 |
| Mesencephalon - Retinal Arborization Field 9 (AF9) | −1596.66 |
| Rhombencephalon - Vmat2 Cluster 4 | −1654.06 |
| Rhombencephalon - Isl1 Stripe 1 | −1848.66 |
| Rhombencephalon - MiV2 | −2189.92 |
| Rhombencephalon - ROM1 | −2492.86 |
| Rhombencephalon - Gad1b Stripe 3 | −2670.18 |
| Rhombencephalon - 6.7FDhcrtR-Gal4 Stripe 4 | −2906.2 |
| Rhombencephalon - Neuropil Region 5 | −3059 |
| Rhombencephalon - Eminentia Granularis | −3571.31 |
| Rhombencephalon - Lobus caudalis cerebelli | −3657.53 |
| Rhombencephalon - MiD3 | −3729.26 |
| Ganglia - Olfactory Epithelium | −4226.91 |
| Rhombencephalon - Vglut2 Stripe 4 | −4345.41 |
| Ganglia - Lateral Line Neuromast SO2 | −4603.11 |
| Diencephalon - Pretectum | −4991.41 |
| Rhombencephalon - Vmat2 Cluster 3 | −5014.93 |
| Diencephalon - Olig2 Band | −5181.96 |
| Rhombencephalon - | −5209.17 |
| Spinal Cord - Gad1b Stripe 1 | −5273.71 |
| Rhombencephalon - MiR2 | −5331.18 |
| Rhombencephalon - Glyt2 Stripe 2 | −5624.12 |
| Rhombencephalon - 6.7FDhcrtR-Gal4 Cluster 1 | −5704.76 |
| Rhombencephalon - Gad1b Cluster 1 | −5758.96 |
| Diencephalon - Hypothalamus Vglut2 Cluster 3 | −6030.64 |
| Rhombencephalon - Gad1b Cluster 10 | −6071.82 |
| Rhombencephalon - Vmat2 Cluster 2 | −6192.22 |
| Rhombencephalon - Valvula Cerebelli | −6413.58 |
| Rhombencephalon - VII Facial Motor and octavolateralis efferent neurons | −6538.98 |
| Rhombencephalon - Glyt2 Cluster 13 | −6786.51 |
| Rhombencephalon - Gad1b Cluster 15 | −7078.93 |
| Rhombencephalon - Cerebelluar-Vglut2 enriched areas | −7195.48 |
| Rhombencephalon - Gad1b Cluster 4 | −7260.67 |
| Diencephalon - Diffuse Nucleus of the Intermediate Hypothalamus | −7484.74 |
| Rhombencephalon - Gad1b Cluster 14 | −7520.1 |
| Spinal Cord - Vglut2 Stripe 2 | −7699.64 |
| Rhombencephalon - Oxtl Cluster 1 Sparse | −7763.7 |
| Rhombencephalon - 6.7FDhcrtR-Gal4 Stripe 3 | −7861.68 |
| Rhombencephalon - Area Postrema | −8077.35 |
| Mesencephalon - Tecum Neuropil | −8979.3 |
| Rhombencephalon - Rhombomere 6 | −9287.98 |
| Spinal Cord - Neurons with descending projections labelled by spinal | −9470.36 |
| backfills | |
| Rhombencephalon - Vglut2 Stripe 2 | −9628.79 |
| Rhombencephalon - Olig2 Stripe | −9679.08 |
| Mesencephalon - Ptf1a Cluster | −9771.8 |
| Mesencephalon - Torus Semicircularis | −9872 |
| Rhombencephalon - Otpb Cluster 6 | −10114.1 |
| Rhombencephalon - Gad1b Stripe 2 | −10145.3 |
| Mesencephalon - Tectum Stratum Periventriculare | −10882.9 |
| Diencephalon - Hypothalamus 6.7FRhcrtR-Gal4 cluster 2 | −10979.6 |
| Rhombencephalon - Vmat2 Stripe2 | −10984.8 |
| Spinal Cord - Glyt2 Stripe | −11146.3 |
| Spinal Cord - Vmat2 Stripe2 | −11504.9 |
| Spinal Cord - Olig2 Stripe | −11559.7 |
| Rhombencephalon - VII′ Facial Motor and octavolateralis efferent | −11669.4 |
| neurons | |
| Rhombencephalon - Raphe - Inferior | −11719.4 |
| Spinal Cord - Gad1b Stripe 2 | −11940.3 |
| Mesencephalon - | −12131.2 |
| Mesencephalon - NucMLF (nucleus of the medial longitudinal fascicle) | −12589.4 |
| Spinal Cord - Vglut2 Stripe 1 | −12590.5 |
| Rhombencephalon - Qrfp neuron cluster sparse | −12770 |
| Rhombencephalon - Gad1b Cluster 2 | −12838.9 |
| Rhombencephalon - Small cluster of TH stained neurons | −12916.1 |
| Rhombencephalon - Gad1b Cluster 3 | −12980.5 |
| Rhombencephalon - Vglut2 cluster 3 | −13124.5 |
| Spinal Cord | −13379.6 |
| Spinal Cord - Dorsal Sparse Isl1 cluster | −13869.8 |
| Rhombencephalon - Gad1b Cluster 7 | −14002.8 |
| Rhombencephalon - Raphe - Superior | −14014 |
| Rhombencephalon - Vglut2 Stripe 1 | −14030.8 |
| Diencephalon - Retinal Arborization Field 6 (AF6) | −14062.4 |
| Rhombencephalon - 6.7FDhcrtR-Gal4 Stripe 2 | −14201.4 |
| Spinal Cord - Isl1 stripe - motorneurons | −14919.8 |
| Spinal Cord - Vmat2 Stripe 1 | −15987.8 |
| Rhombencephalon - Caudal Ventral Cluster Labelled by Spinal Backfills | −16124.8 |
| Rhombencephalon - Rhombomere 7 | −16482.9 |
| Rhombencephalon - Oculomotor Nucleus nIV | −16552.8 |
| Spinal Cord - 6.7FDhcrtR-Gal4 Stripe | −16848.3 |
| Rhombencephalon - Cerebellar Neuropil 1 | −17217.1 |
| Mesencephalon - Tegmentum | −17275 |
| Rhombencephalon - Glyt2 Stripe 1 | −17296.4 |
| Rhombencephalon - Gad1b Stripe 1 | −17753 |
| Rhombencephalon - Noradrendergic neurons of the Interfascicular and | −17975.9 |
| Vagal areas | |
| Rhombencephalon - 6.7FDhcrtR-Gal4 Cluster 4 | −18034.5 |
| Rhombencephalon - Vmat2 Stripe1 | −18644.6 |
| Rhombencephalon - Neuropil Region 3 | −18941.6 |
| Rhombencephalon - Glyt2 Cluster 2 | −19040.1 |
| Rhombencephalon - Gad1b Cluster 11 | −19340.2 |
| Rhombencephalon - 6.7FDhcrtR-Gal4 Cluster 3 | −19534.9 |
| Rhombencephalon - Neuropil Region 2 | −19822.6 |
| Rhombencephalon - Ptf1a Stripe | −20843.2 |
| Diencephalon - Migrated Area of the Pretectum (M1) | −20927 |
| Rhombencephalon - CaD | −21077.6 |
| Spinal Cord - Vglut2 Stripe 3 | −21498.9 |
| Mesencephalon - Oculomotor Nucleus nill | −21691.4 |
| Rhombencephalon - X Vagus motorneuron cluster | −22874.8 |
| Spinal Cord - Neuropil Region | −23691.3 |
| Rhombencephalon - Gad1b Cluster 20 | −23696.4 |
| Mesencephalon - Medial Tectal Band | −24021.2 |
| Rhombencephalon - Glyt2 Cluster 4 | −25218.5 |
| Rhombencephalon - Inferior Olive | −25341 |
| Ganglia - Lateral Line Neuromast SO1 | −29011.5 |
| Rhombencephalon - CaV | −30272.6 |
| Ganglia - Lateral Line Neuromast D2 | −30431.6 |
| Rhombencephalon - Gad1b Cluster 12 | −30696.7 |
| Mesencephalon - Retinal Arborization Field 8 (AF8) | −31776.1 |
| Rhombencephalon - 6.7FDhcrtR-Gal4 Stripe 1 | −32120.8 |
| Mesencephalon - Vglut2 cluster 1 | −32644.2 |
| Mesencephalon - Retinal Arborization Field 7 (AF7) | −37025.7 |
| Mesencephalon - Oxtl Cluster Sparse | −37688.9 |
| Rhombencephalon - Vglut2 cluster 4 | −38787.3 |
| Mesencephalon - Otpb Cluster | −44114.1 |
| Mesencephalon - Sparse 6.7FRhcrtR cluster | −45346.8 |
| Diencephalon - Retinal Arborization Field 5 (AF5) | −48696.6 |
| Rhombencephalon - Lateral Reticular Nucleus | −54715 |

AS1 specifically engages D1 receptor dopaminergic circuitry. The enrichment of brain regions containing dopaminergic neurons or receiving dopaminergic innervation (i.e., clusters within the zebrafish basal ganglia equivalent) in the activity profiles of fish concurrently exposed to AS1 and noxious heat prompted further exploration of the hypothesis that AS1 engages dopaminergic circuits. To accomplish this, the behavioral aversion assays were repeated following pharmacological manipulation of dopamine receptor signaling. Like mammals, zebrafish possess multiple dopamine receptors, and the analogs of mammalian receptors most associated with valence assignment, the D1 and D2 receptors, were targeted. In mammals, these dopamine receptor subtypes are largely expressed on non-overlapping populations of striatal medium spiny neurons (MSNs), and play opposing roles in valence assignment and reward processing—in brief, stimulation of D1 receptors has been shown to facilitate reward and positive valence assignment, whereas D2 receptor activation elicits aversion (Verharen et al., Neuropsychopharmacology. 2019; 44:2195-2204; Kravitz et al., Nat. Neurosci. 2012; 15:816-818; Surmeier et al., Trends Neurosci. 2007; 30:228-235).

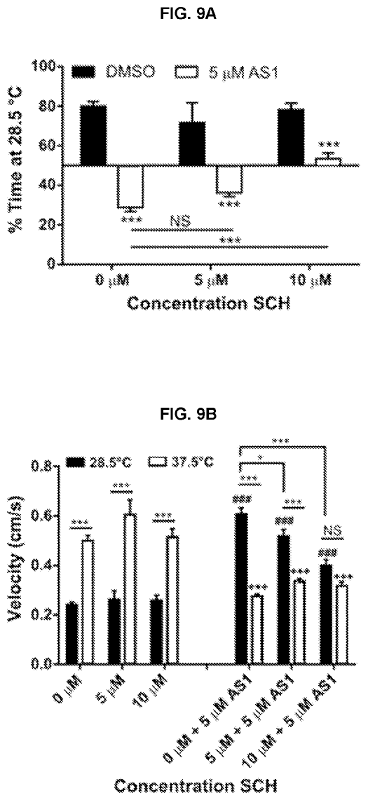

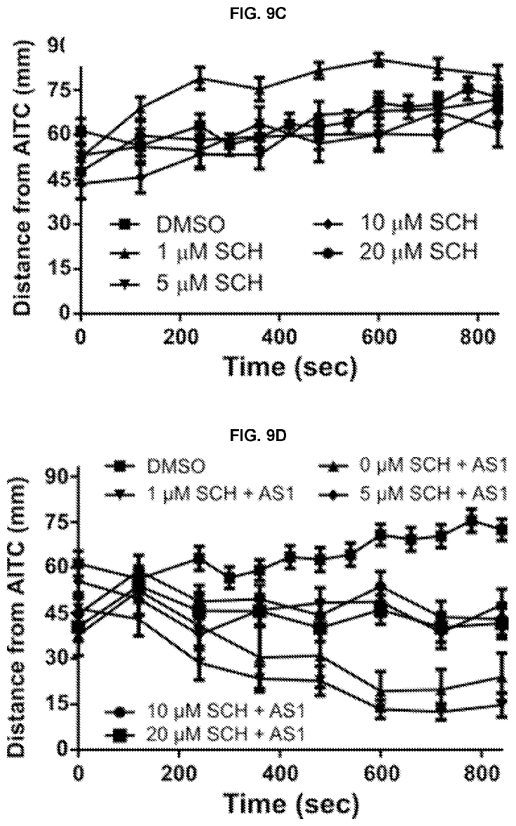

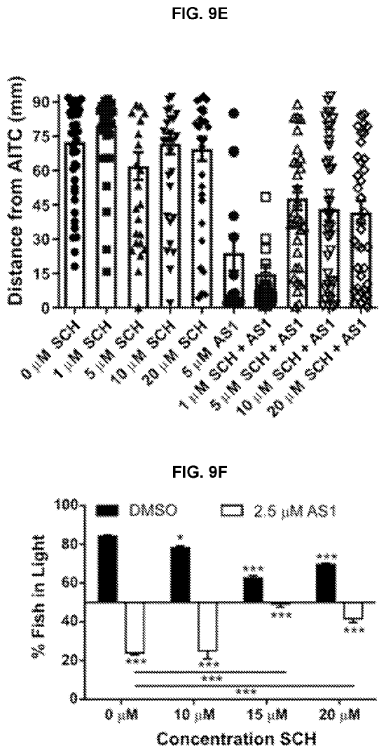

Remarkably, inhibition of D1 activity with the selective D1 antagonist SCH23390 (10 μM) partially restored aversion to noxious heat in the presence of AS1 without eliciting effects at baseline (FIG. 9). Additionally, SCH23390 was able to decrease the heightened velocity at 28.5° C. induced by AS1 in a dose-dependent manner (FIG. 9). While SCH23390 by itself had no effect upon aversive behavior in response to the chemical irritant AITC at multiple concentrations, it attenuated AS1-mediated attraction to AITC in a dose-dependent manner (FIG. 9). These effects extended to the light-dark preference assay, with intermediate-high doses blocking AS1-induced preference for the dark (FIG. 9). While high concentrations of SCH23390 appear to significantly decrease light preference in the absence of AS1, it seems unlikely that this would explain the observed attenuation of the AS1-induced dark preference following treatment with SCH23390. However, it may be possible that SCH23390-mediated D1 inhibition may be blunting phototaxic choice in general.

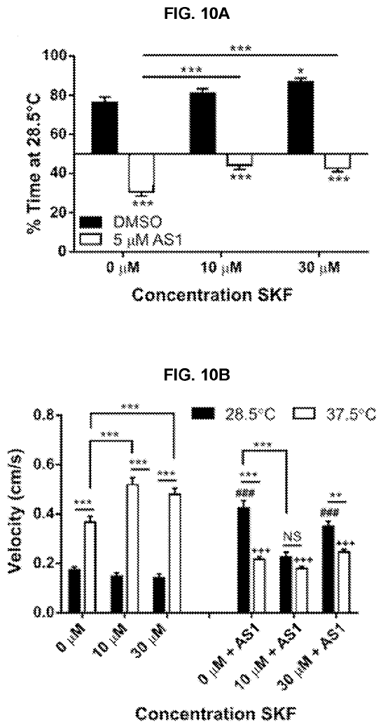

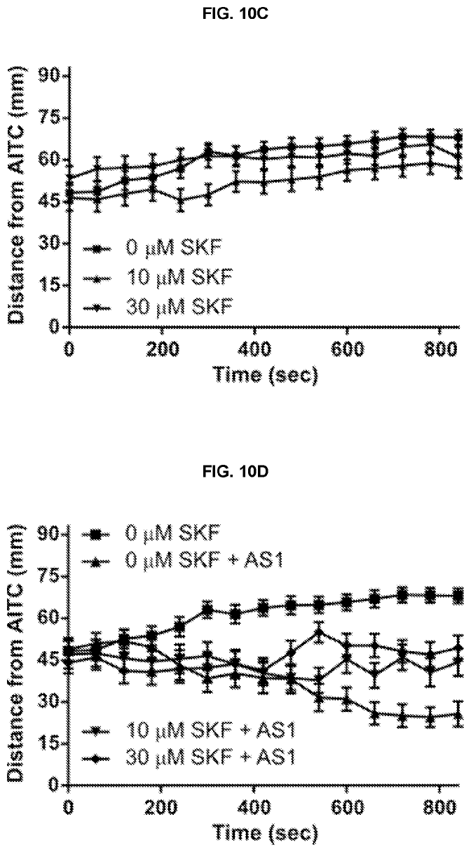

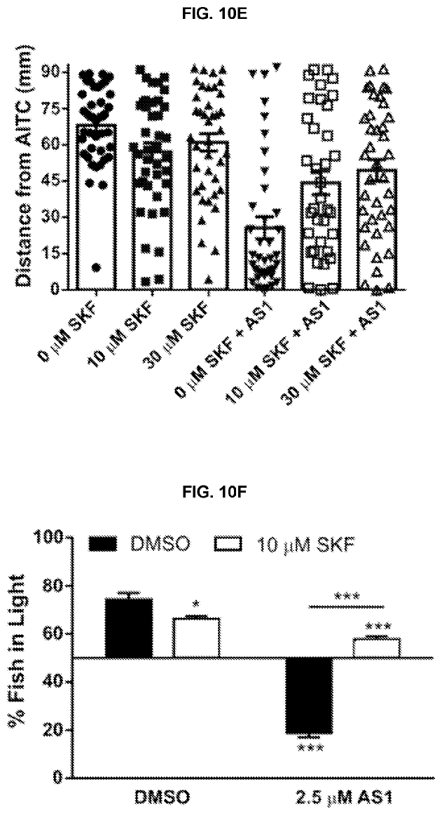

Activating D1 receptors may also dampen the effects of AS1 in these place preference assays, as D1 receptors would no longer be selectively activated in the presence of noxious stimuli. Indeed, application of the selective D1 receptor agonist SKF82958 blunted AS1-evoked preference for aversive stimuli in all assays. Both 10 μM and 30 μM SKF significantly attenuated the amount of time AS1-treated fish spent at 37.5° C. in the temperature preference assay (FIG. 10). Much like SCH23390, SKF82958 also reduced the swimming velocity of AS1-treated zebrafish in the 28.5° C. zone, although swimming velocity in the 37.5° C. zone remained unaffected (FIG. 10). SKF82958 by itself had little effect upon aversion to AITC (FIG. 10), but did block AS1-induced preference to this chemical irritant (FIG. 10). Similarly, exposure to the D1 receptor agonist reversed dark preference in fish that were treated with AS1 (FIG. 10).

Interestingly, neither treatment with the D1R agonist nor antagonist was able to completely ablate the effects of AS1 and restore thermal, chemical, and dark aversion back to baseline levels. At most, only weak aversion or neutral preference was observed, even at the highest concentrations of SCH23390 and SKF82958 tested. It is possible that AS1 may elicit analgesia separately from its effects upon valence assignment, and via a dopamine-independent mechanism.

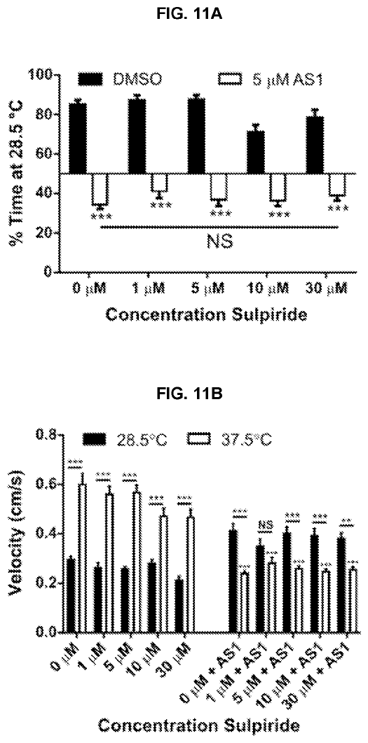

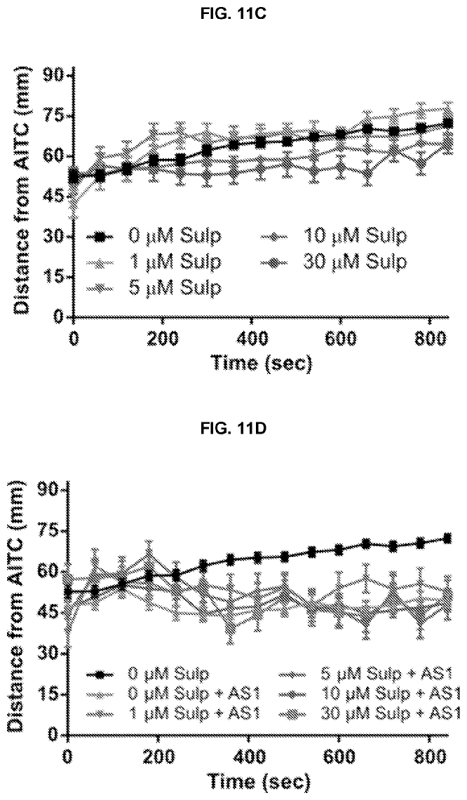

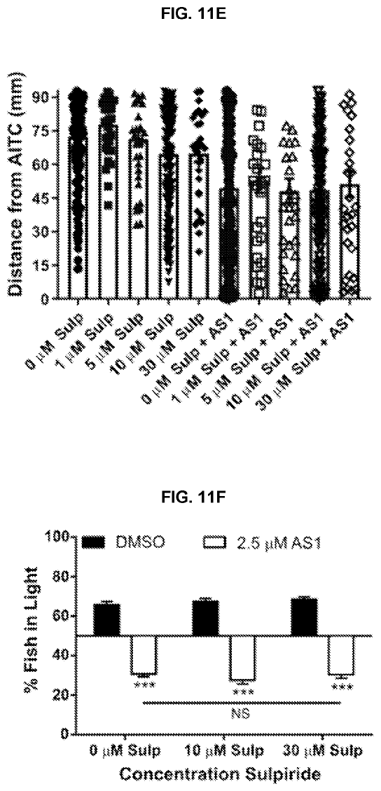

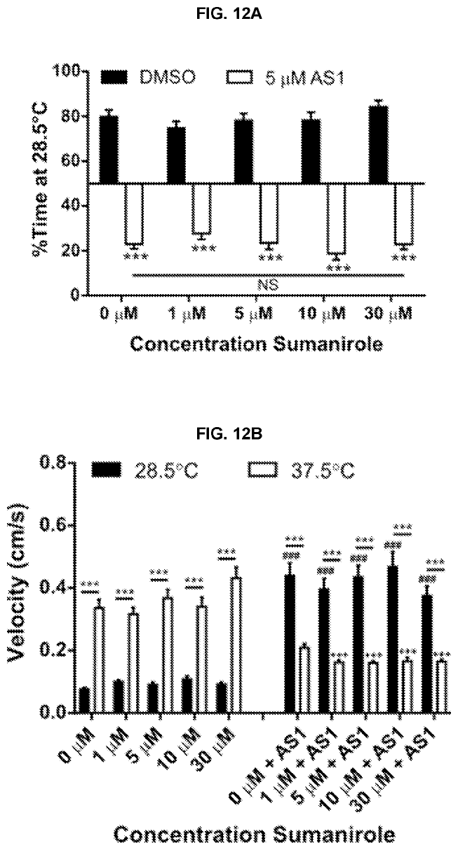

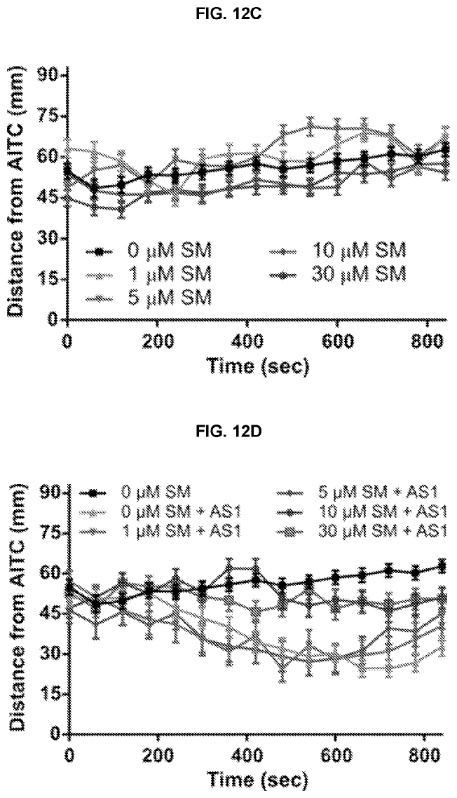

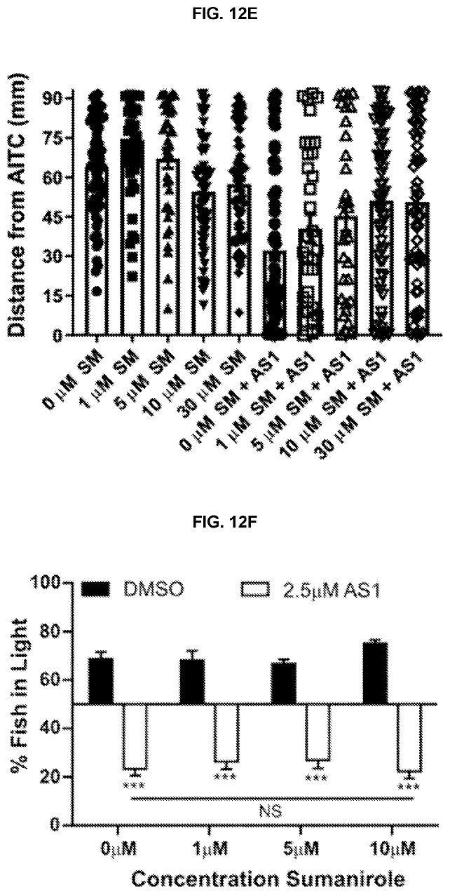

Treatment with the selective D2 receptor antagonist sulpiride alone had no effect upon the behavior of 6 dpf larval zebrafish in the temperature choice assay, AITC aversion assay, or light/dark preference assay (FIG. 11). Unlike the D1 receptor antagonist, application of sulpiride to AS1-treated fish had no effect upon AS1-induced reversal of temperature choice, swimming velocity in the 37.5° C. zone, AITC aversion, or light preference (FIG. 11). Similarly, the selective D2 receptor agonist sumanirole maleate had no effect either alone or upon AS1-mediated attraction to noxious heat or dark stimuli (FIG. 12). Interestingly, while this drug had little effect alone in the chemical aversion assay, it did seem to attenuate AS1-mediated attraction to AITC, but only at higher concentrations (FIG. 12). Without being limited by mechanism or theory, it is possible that in this particular scenario, activating the D2 receptor is mimicking the effects of SKF—by activating the D2 receptor, likely inhibiting neurons in the “off” pathway, perhaps creating the sensation of reward regardless of environmental context.

### Materials and Methods

Zebrafish Husbandry. Adult Zebrafish (Danio rerio) were raised with constant filtration, temperature control (28.5±2° C.), illumination (14 hr:10 hr light-dark cycle, lights on at 9:00 AM), and feeding. All animals were maintained in these standard conditions and the Institutional Animal Care and Use Committee approved all experiments. Adult zebrafish not used in behavioral experiments were bred in spawning traps (Thoren Caging Systems, Hazelton, PA) from which embryos were collected. Larval zebrafish were raised in petri dishes (Fisher Scientific, Hampton, NH) of E2 medium with no more than 50 embryos per dish at 28.5±1° C. in an incubator (Sanyo). Embryos were staged essentially as described (Kimmel et al., Dev Dyn. 1995; 203(3):253-310) and kept until 6 dpf.

Chemicals. The following chemicals were procured from Millipore-Sigma: SCH23390 hydrochloride (cat #: D054-5MG), sulpiride (cat #: S7771-5G), sumanirole maleate (cat #: S7771-5G), allyl isothiocyanate (cat #: 377430-100G), and caffeine (cat #: C0750-5G). SKF 82958 hydrobromide (cat #57-191-0), buspirone hydrochloride (cat #09-621-00), and DMSO (cat #D128-4) were purchased from Fisher Scientific. The naloxone hydrochloride (cat #: 0599) was purchased from R&D Systems Inc (a Bio-Techne brand). Diazepam (Hospira, Inc.) was obtained from the Drug Services office at the University of Washington. All other reagent sources are noted in their respective sections.

### Behavioral Assays.

Thermal preference assay. Thermal preference assays were performed as previously described (Curtright et al., PLoS One. 2015; 10:1-18). In brief, individual, randomly selected 5-6 dpf larval zebrafish were caught in 50-100 μL E2 media using a p200 micropiette equipped with specialized large orifice 200 μL pipet tips (USA Scientific, cat #: 1011-8000) and deposited individually into wells of custom-made choice testing plates (one larva per well). These plates were made by machining 32 oval shaped, 20 mm by 8 mm arenas out of a 5 cm×39 cm rectangle of plastic, which was bonded to 0.002 in thick aluminum shim (ShopAid, cat #40002) using a waterproof adhesive (DAP, cat #: 00688). Once an entire plate was loaded with fish, the appropriate incubation solution was added. For all incubations, choice testing plates were returned to the 28.5° C. incubator. Following incubation in all experiments, the choice testing plate was transported to a dual solid-state heat/cool plate (AHP-1200° C. P; Teca) and centered such that half of each arena was positioned over each side of the heat/cool plate. One side of the heat/cool plate was always maintained at rearing temperature (28.5° C.), while the temperature of the other side was adjusted according to the experiment. Locomotor behavior was recorded using a Canon high-definition video camcorder suspended at a fixed position above the choice testing plate. Each trial was four minutes in duration.

For single-incubation assays (e.g. testing single chemicals), larval zebrafish were caught in 100 μL E2 media and 100 μL of the control (2% DMSO) or test chemical at 2× concentration was added to each well to achieve the final desired concentration. Choice testing plates were placed in the 28.5° C. incubator to incubate for 10 minutes before the filmed trial. For double-incubation assays (e.g., testing the impact of various chemicals on the effects of AS1), zebrafish were caught in 50 μL E2 media and 50 μL of the first incubation solution (2× control or test chemical) was added to each well, and plates were incubated at 28.5° C. for ten minutes. 100 μL of the second incubation solution (1× control or test chemical +/−2× AS1) was added to each well, and the plate was incubated at 28.5° C. for another ten minutes before beginning the filmed trial. For the sensitized thermal aversion assay in the initial drug screen, larvae were pre-incubated in the appropriate drug solutions for ten minutes, and allyl isothiocyanate (AITC; Sigma, cat #: 377430) was added to achieve a final concentration of 0.5 μM AITC immediately before filming. The final DMSO concentration in all solutions was 1%.

Chemical attraction/aversion assays. The agarose attraction/aversion assay was adapted from previously described experiments (Jordi et al., Sci. Adv. 2018; 4:1-15). For the AITC aversion assay, AITC and DMSO were added to molten 0.8% agarose to achieve a final concentration of 100 mM AITC and 2% DMSO. For the AS1 attraction/aversion assay, AS1 and DMSO were added to 0.8% molten agar to achieve final concentrations of 50 mM and 2%, respectively. To construct the test chambers, the lids of 10×10 cm square petri dishes (insert cat #and company) were lined on four sides with either the test (chemical-containing) or plain agarose (300 μL per side) and allowed to solidify. For all experiments, 30-40 randomly selected 6 dpf zebrafish were caught with a 10 mL pipette pump (Bel-Air Products, cat #: 13683C) equipped with a glass wide-bored Pasteur pipet (Fisher Scientific, cat #: 22-037-540) and deposited into a standard 10 cm diameter petri dish (Fisher Scientific, cat #: 07-202-031). As much E2 media as possible was carefully removed using the same pipette. For single incubation experiments, 30 mL of 1× solutions of the control or test chemical was added to each petri dish, and fish were incubated for 10 minutes at 28.5° C. For double incubation experiments, 15 mL of 1× solutions containing control (DMSO) or test chemicals was added to the larvae-containing petri dish. Following a ten minute incubation, 15 mL of the second incubation solution (1× control or test chemical +/−2× AS1) was added to the petri dish, and the fish were incubated for a second ten minute block. The final concentration of DMSO in all solutions was 1%. In all double-incubation experiments, the final concentration of AS1 was 5 μM unless otherwise noted. After completion of the last incubation period, the contents of each petri dish were poured into separate agarose-lined square dishes. Swimming behavior was immediately recorded for 20 minutes using the same high-definition camcorder in the thermal preference assays.

For the SKF attraction/aversion assay, custom 10 cm×10 cm plates using clear resin (Formlabs, cat #: RS-F2-GPCL-04) and a Form 3+3D printer (Formlabs, cat #: PKG-F3-WSVC-BASIC) were designed. Each plate had a 9.5×0.5 cm trough at two opposing ends. Larval zebrafish (N of 30 to 40) were carefully pipetted into the middle of the plate in as little E2 media as possible. 2 mL of the 25 μM SKF solution was deposited into one trough, while 1% DMSO was added to the opposite trough. Just enough E2 media was added to the plate to join the small E2 pool containing larval fish to the contents of each trough, and care was taken to ensure the solution was disturbed as little as possible. Swimming behavior was then recorded for 20 minutes. This format was also used to test the attractiveness of AS1 (25 μM-1 mM) in order to have a better standard of comparison for the SKF. In the case of 1 mM AS1, 10% DMSO was added to the control side given that that was the concentration of DMSO vehicle in that solution.

Light/dark preference (phototaxis) assay. In the light/dark preference assay, randomly-selected 6 dpf larval zebrafish (N of 30 to 40) were carefully pipetted onto a 10 cm square petri dish using a 10 mL pipette pump equipped with a glass wide-bored Pasteur pipet. As much E2 media as possible was carefully removed using the same pipet. For single incubation experiments, 30 mL of 1× solutions of the control or test chemical was added to each petri dish, and fish were incubated for 10 minutes at 28.5° C. For double incubation experiments, 15 mL of 1× solutions containing control (DMSO) or test chemicals was added to the larvae-containing petri dish. Following a ten-minute incubation period, 15 mL of the second incubation solution (1× control or test chemical +/−2× AS1) was added to the petri dish, and the fish were incubated for a second ten minute block. The final concentration of DMSO in all solutions was 1%. In all double-incubation experiments, the final concentration of AS1 was 2.5 μM. These petri dishes were then positioned over a horizontally-oriented computer monitor displaying a PowerPoint presentation. For standard light/dark preference assays, a blank white slide was initially presented for one minute, after which the presentation would automatically advance to a slide in which half of the display was black. The petri dish with larvae was positioned such that exactly half was directly over the dark side, and the other half was directly over the light side. After four minutes, the presentation automatically advanced to a slide in which the black and white halves switched places. A total of five four-minute trials, with the dark/light halves automatically switching position between trials, were recorded. For the gradient phototaxis assay, experiments were performed identically, except that the “dark” half of the powerpoint presentation was one of six shades of gray.

In all experiments, an initial still frame of video was taken during the minute where the blank slide was presented in order to quantify the total number of fish in the experiment. Following this, still frames were taken at 30 second intervals for each trial (T=0, 30, 90, 120, 150, 180, 210, and 240 seconds), and the number of fish present in the light half of the arena were counted. To generate the graphs that looked at swimming patterns over time in a trial (e.g., FIG. 3D), the average percentage of fish in the light at each time point (T=0, T=30, etc.) was calculated across all five trials. To calculate graphs, only counts from the final five time points (t=120-240 seconds) of the last four trials were counted when calculating the average percentage of fish in the light, to allow fish to have time to make a choice.

CaMPARI Neuronal Activity Assay. elav/3:CaMPARI zebrafish in the Casper background were simultaneously exposed to chemical stimuli and a 405 nm light in order to permanently photoconvert active neurons (Fosque et al., Science. 2015; 347(6223):755-760.). Briefly, 6 dpf larval zebrafish were anesthetized with iced E2 medium, immobilized with a harp (Harvard Apparatus, cat #64-0253), and paralyzed by injecting α-bungarotoxin protein (Invitrogen, cat #: B-1601), into the chest cavity using microinjection needles pulled on a Flaming-Brown Micropipette Puller (model P-87, Sutter Instrument Co., Novato, CA) and a Picrosprizter II microinjection apparatus (General Valve Corporation, Fairfield, NJ). Paralyzed fish were then pre-incubated in either 1% DMSO or 5 μM AS1 for 2 min and then immersed in a water bath set to either rearing temperature (28.5° C.) or noxious heat (36.5° C.). Following this incubation period, fish were immediately placed glass-bottomed dishes (Wilco Wells, Netherlands) and placed on the stage of an inverted fluorescent microscope (Olympus, Japan, model Ix81S1F-3) and the larvae were exposed to a 405 nm light for 40 s using MetaMorph software (Molecular Devices, San Jose, CA). Post-exposure fish were removed from the chemical and placed in a petri dish filled with embryo media and tricaine to prevent any future activation of sensory neurons. Immediately prior to imaging, larvae were mounted on coverslips in 1.5% agarose+tricaine in E2 media. TG and surrounding neural tissue were imaged using a 20× lens on an LSM 880 confocal microscope (Zeiss, Germany). Zen Black software was used to scan through the entire TG. Images were examined for photoconverted (red-labeled) neurons, and totals were established for each TG in each condition.

pERK Immunolabeling. 6 dpf larval zebrafish (N 10-20) in the Casper background were placed into 5 mL microcentrifuge tubes (VWR, cat #: 10015-792) with either 1% DMSO, 2.5 μM AS1, or 5 μM AS1. Depending upon the experimental condition tested, these tubes were placed in either a 28.5° C. or 37.5° C. water bath for 15 minutes (Wee et al., Nat. Neurosci. 2019; 22:1477-1492). Following the 15 minute exposure, fish were immediately anesthetized with tricaine and fixed in 4% paraformaldehyde/0.25% Triton-X for 20-24 hours at 4° C. Following fixation, antibody labeling for both total ERK (tERK) and phosphorylated ERK (pERK) was performed as previously described (Wee et al., Nat. Neurosci. 2019; 22:1477-1492). In brief, larval zebrafish were washed with 0.25% PBT (1× PBS with 0.25% Triton-X) 2-3 times, incubated in 150 mM Tris-HCl (pH 9) at 70° C. for 15 minutes, rinsed with PBT, and incubated in 0.05% Trypsin-EDTA for 45 on ice. Samples were then blocked in blocking buffer (1× PBS, 0.3% Triton-X, 10% goat serum) at room temperature on a rocker for at least one hour. The larvae were then incubated in a primary antibody solution (1:500 rabbit monoclonal Phospho-p44/42 MAPK (Erk1/2) (Thr202/Tyr204) (D13.14.4E) XP and 1:500 mouse monoclonal p44/42 MAPK (Erk1/2) (L34F12), Catalog #4370S and Catalog #4696S from Cell Signaling Technologies, Inc., respectively) at 4° C. on a rocker for up to three days. After this, samples were washed three times in PBT and incubated in a secondary antibody solution (AlexaFluor goat anti-mouse 488 and AlexaFluor goat anti-rabbit 568, both 1:500, cat #s A32723 and A-11011 from Invitrogen, respectively) at 4° C. on a rocker shielded from light for 24 hours. Samples were then washed 3 times in PBT and stored in 50% glycerol/1× PBS at 4° C. until imaging.

Confocal Imaging and MAP-Mapping. Fixed, pERK/tERK immunolabeled zebrafish were dorsally mounted in 1.5% low-melt agarose to facilitate imaging. Entire brains were imaged using a 10× air objective on a Zeiss LSM 880 confocal microscope (5 μm step size). In order to map experimental brains onto a reference brain, these composite confocal z-stacks were first split into individual channels in ImageJ, and each of those stacks was saved as an .nrrd file. Image stacks in this file format were then registered to a reference brain using the CMTK registration tool (GUI plugin courtesy of the Jeffries lab) on ImageJ (Randlett et al., Nat. Methods. 2015; 12:1039-1046; Cachero et al., Curr. Biol. 2010; 20:1589-1601). In the CMTK registration GUI, the registration parameters were set to “Cachero, Ostrovsky 2010”, and -awr 0102-X 52-C 8-G 80-R 3-A-accuracy 0.4& #39; -W-accuracy 1.6 were used as further registration parameters. Registered stacks were then individually visually inspected to ensure that they had registered correctly, and all error-free stacks were then downsampled (“smoothed”) using a previously-developed ImageJ script (PrepareStacksForMAPMapping.ijm) (Randlett et al., Nat. Methods. 2015; 12:1039-1046) and sorted into individual folders based upon condition. Each experimental condition (AS1 Only, Heat Only, and AS1+Heat) was then compared to the Control Group using the MakeTheMAPMap.m Matlab script. One of the output files for this script, a SignificantDeltaMedians file, was then used as an input to run the ZBrainAnalysisOfMAPMaps.m Matlab script, which generated excel files showing which ROIs were significantly upregulated or downregulated from each comparison. Net activation for each ROI was determined by subtracting the negative signal from the positive signal. To determine neural activity specific to AS1 treatment in the context of noxious heat, the AS1 Only and Heat Only signals were subtracted from the AS1+Heat values for each ROI.

Statistical Analyses. All statistical analyses were performed using GraphPad Prism software.

### (v) Closing Paragraphs

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1.-33. (canceled)

34. A composition comprising:
at least one of:
4-(Propan-2-yl)-N-(pyridin-4-yl)benzamide,
4-propyl-N-4-pyridinylbenzamide,
4-isopropyl-N-3-pyridinylbenzamide, or
N-(4-isopropylphenyl)-3,5-dimethyl-4-isoxazolecarboxamide; and
a pharmaceutically acceptable carrier.

35. The composition of claim 34, wherein the composition comprises the 4-(Propan-2-yl)-N-(pyridin-4-yl)benzamide.

36. The composition of claim 35, formulated for oral delivery or injection.

37. The composition of claim 36, wherein the composition formulated for oral delivery is a tablet, pill, dragee, capsule, liquid, gel, syrup, slurry, or suspension.

38. A method of treating a subject in need thereof comprising administering a therapeutically effective amount of the composition of claim 34 to the subject, thereby treating the subject in need thereof.

39. The method of claim 38, wherein the administering occurs when the subject
is experiencing pain and the treating reduces perception of pain or
is experiencing an aversive reaction to a stimulus and the treating reduces the aversive reaction to the stimulus.

40. The method of claim 38, wherein the administering occurs
before the subject is expected to experience pain and the treating reduces the expected experience of pain, or
before the subject is expected to experience an aversive reaction to a stimulus and the treating reduces the experience of the aversive reaction to the stimulus.

41. The method of claim 38, wherein the subject has anxiety, an anxiety-related disorder, depression, or a post-traumatic stress disorder.

42. The method of claim 38, wherein the subject is a mammal.

43. The method of claim 38, wherein the administering is oral, sublingual, subcutaneous, intravenous, intradermal, intraarterial, intranodal, intralymphatic, intraperitoneal, intralesional, topical, or intramuscular.

44. A method of activating a portion of a brain region in a subject comprising administering a therapeutically effective amount of the composition of claim 34 to the subject, thereby activating the portion of the brain region.

45. The method of claim 44, wherein the administering occurs when the subject is experiencing pain or an aversive reaction to a stimulus.

46. The method of claim 44, wherein the administering occurs before the subject is expected to experience pain or before the subject is expected to experience an aversive reaction to a stimulus.

47. The method of claim 44, wherein the subject has anxiety, an anxiety-related disorder, depression, or post-traumatic stress disorder.

48. The method of claim 44, wherein the subject is a mammal.

49. The method of claim 44, wherein the administering is oral, sublingual, subcutaneous, intravenous, intradermal, intraarterial, intranodal, intralymphatic, intraperitoneal, intralesional, topical, or intramuscular.

50. The method of claim 44, wherein the brain region is a subpallium, within a telencephalic region, or within a diencephalon region.

51. The method of claim 50, wherein the brain region within the telencephalic region is a basal ganglia, a striatum, an extended amygdala, or an amygdala.

52. The method of claim 50, wherein the brain region within the diencephalon region is a posterior tuberculum or hypothalamus.

53. The method of claim 44, further comprising detecting activation of the portion of the brain region by imaging.
